(19) United States
(12) Patent Application Publication
(10) Pub. No.: US 2008/0287774 A1
(43) Pub. Date: Nov. 20, 2008

(54) Magnetic Resonance Imaging and Spectroscopy Means and Methods Thereof

(76) Inventor: Rachel Katz-Brull, Rehovot (IL)

Correspondence Address: Fleit Gibbons Gutman Bongini & Bianco PL, 21355 EAST DIXIE HIGHWAY, SUITE 115, MIAMI, FL, 33180, US

(21) Appl. No.: 12/092,763

(22) PCT Filed: Nov. 2, 2006
(86) PCT No.: PCT/IL06/01268

§ 371 Date: May 23, 2008

(30) Foreign Application Priority Data
Nov. 6, 2005 (IL) 171790

(51) Int. Cl. A61B 5/055

(52) U.S. Cl. 600/414; 424/9.1; 424/9.37

(57) ABSTRACT

The present invention discloses neurochemical agents and biochemical agents for human or mammalian neuro- and body-metabolic imaging, comprising chemicals involved in neuronal or glial function, neuromodulatory processes in the brain of said human or mammalian, vascular function, or organ specific metabolic processes; said neurochemical and biochemical agents are labeled with stable isotopes selected from a group including carbon-13, nitrogen-15, deuterium, fluorine-19 or a combination thereof in predetermined positions, so as to enhance the detectability of the agents and their metabolic successors.

### FIELD OF THE INVENTION

This invention generally relates to MRI and spectroscopy means and methods thereof, and especially to magnetic resonance imaging and spectroscopy, and brain function as related to metabolism, psychobiology, psychiatry, neurology, and neurodegeneration.

### BACKGROUND OF THE INVENTION

People suffering from psychiatric or neurodegenerative diseases are thought to have altered levels of some of the chemical messengers in the brain, called neurotransmitters and neuromodulators. In depression, the two principal chemical compounds involved are noradrenaline and serotonin. Nerve cells in the brain constantly produce, release and reabsorb serotonin. Lower levels of serotonin are thought to lead to the transmission of faulty messages and to be responsible for some of the symptoms of depression. Drugs such as selective serotonin reuptake inhibitors (SSRIs) increase the levels of noradrenaline and serotonin. This increased brain activity is intended to improve mood. SSRIs are now the most commonly prescribed type of antidepressant drugs. This group includes: fluoxetine; sertraline; paroxetine; fluvoxamine; citalopram; escitalopram; venlafaxine; nefazodone; and mirtazapine. Although not a SSRI, bupropion is a popular antidepressant. These drugs are prescribed by physicians, neurologists, and psychiatrists. After the patients had begun taking a medication, their health is closely monitored throughout the time the patient is taking the medicine. However, as in the case of any drug there are side effects and cases where the patient's symptoms are not alleviated within a reasonable amount of time. The latter usually switch to another medicine but the time allowed for evaluation of drug efficacy before switching to another drug regime is weeks to months. The side effects of SSRIs can be mild to serious including: nausea, difficulty sleeping, drowsiness, anxiety, nervousness, weakness, loss of appetite, tremors, dry mouth, sweating, decreased sex drive, impotence, and the emergence of suicidality. Despite the wide use of SSRIs, the exact biochemical effect of the drug on the individual's brain is not known and can not be quantified with existing technology. The lack of such knowledge has been specifically poignant in the case of depressed children and adolescents who were treated with SSRI and were reported to have developed suicidal behavior. However, despite an overwhelming need for better means to quantify the effects of psychiatric drugs on the brain, in situ, the technological means for doing so had not surfaced.

By far, the most widely used evaluation of brain function in humans (and drug efficacy) is being carried out by neurologists and psychiatrists testing the end results of brain function by quantifying human cognition and behavior according to neurological and psychiatric tests and scales. Examples of such scales include: 1) DSM—Diagnostic and Statistical Manual of Mental Disorders, a manual, published by the American Psychiatric Association, that provides standardized criteria for the diagnosis of psychiatric conditions. The current edition, published in 1994 is the 4th edition, called DSM-IV; 2) CIBIC plus—Clinician's Interview Based Assessment of Change-Plus; 3) MMSE—Mini-Mental State Examination; 4) QoL—patient rated Quality of Life; 5) ADAS—Alzheimer's Disease Assessment Scale; 6) CDR-SB—Clinical Dementia Rating Scale-Sum of the Boxes; 7) CNS—The Canadian Neurological Scale, for assessing neurological function in conscious stroke patients; 8) Montgomery-Asberg Depression Rating Scale (MADRS); 9) Hamilton Rating Scale for Depression (HAM-D); 10) Young Mania Rating Scale (YMRS); 11) Brief Psychiatric Rating Scale (BPRS); and 12) Mini-International Neuropsychiatric Interview (based on DSM-IV criteria).

It is apparent to physicians of the skill that there are numerous other scales and tests to investigate the brain's function by investigating human responses to stimuli, human behavior, and bodily functions. Also, there are several interactive computer software products that are aimed at digitally scaling brain function. Despite their usefulness in diagnosis and in treatment monitoring, such tests do not provide a direct quantifiable biochemical measure of brain activity.

In addition to affective disorders, the levels of serotonin are also related to the serotonin syndrome (or hyperserotonemia) which is a hyperserotonergic state, that is an excess of 5-HT (serotonin) in the central nervous system. It is usually associated with high doses of serotonergic drugs, when combinations of serotonergic agents are used together, or when antidepressants are changed without an adequate washout period between drugs. Less frequently it can also be caused by moderate dosage of a single serotonergic drug, or in combination with non-serotonergeric drugs such as oxycodone, erythromycin, or St. John's Wort.

Serotonin syndrome is rare, but it is a serious, potentially life-threatening medical condition. However there is no lab test for the condition, so diagnosis is by symptom observation. It may go unrecognized because it is often mistaken for a viral illness, anxiety, neurological disorder or worsening psychiatric condition. Clinicians must differentiate between serotonin syndrome and Neuroleptic malignant syndrome, which has similar symptoms. Therefore, the ability to monitor directly the levels of serotonin in the brain may provide a non-invasive test for Serotonin syndrome.

Another example of a brain disease that is treated by drugs which are targeted to affect the metabolism of a neuromodulator is Alzheimer's disease (AD). Alzheimer's disease (AD) is the commonest cause of dementia affecting older people. The symptoms of AD are caused by a continuous loss of neurons and synapses. The current generation of agents used in the treatment of AD consists mostly of acetylcholinesterase (AChE) inhibitors. They act by partially delaying the breakdown of acetylcholine (ACh), a neurotransmitter which is deficient in the brain of patients with AD. The effects of this pharmacologic intervention are symptomatic and compensatory.

Tacrine, the first of the cholinesterase inhibitors to undergo extensive trials for this purpose, was associated with significant adverse effects including hepatotoxicity. Other cholinesterase inhibitors, including rivastigmine, have superior properties in terms of specificity of action and low risk of adverse effects. Ultimately, the benefits of such therapy decline as the neurodegenerative process progresses. Placebo-controlled clinical trials exploring the efficacy and safety have shown that the effects of AChE inhibitors are dose-dependent. As a group, patients receiving high-dose regimens show a slight increase in cognitive function which reaches a maximum after three to six months. This contrasts with the cognitive deterioration observed in patients on placebo. Positive changes in cognition are less prominent in patients receiving low-dose regimens. Improvements in activities of daily living (ADL) are more difficult to assess. In this domain, the average patient receiving a high dose of an AChE inhibitor may exhibit no significant improvement. However, signs and symptoms of AD decline at a slower rate than placebo.

In terms of group means, the effects of AChE inhibitors on cognition and ADL are best described as a stabilization rather than a dramatic improvement. Group means provide little information on the likelihood of treatment outcome in individual patients. Controlled trials with AChE inhibitors have consistently shown individual outcome to be highly variable. On standard scales such as the Alzheimer's Disease Assessment Scale cognitive subscale (ADAS-Cog), a significant proportion of patients respond with considerably higher scores than average, whereas a minority do not benefit from the treatment. If a patient does not respond to an AChE inhibitor, alternative treatments may include nootropics (e.g. piracetam), calcium channel blockers (e.g. nimodipine), glutamate modulators (e.g. memantine), and selegiline. The individual response to these drugs varies considerably.

As in the case of SSRIs, there is no available test to directly determine the effects of AChE inhibitors within the individual's brain, non-invasively. Because of the lack of such a test, and because the efficiency of these drugs can be evaluated only after several weeks or months, it is not uncommon that patients are loosing valuable time in which the disease progresses irreversibly and is not stabilized because the patient is being given a treatment that is inefficient to them. The progress of AD contributes significantly to its societal and economic burden.

Dopamine is another important neuromodulator. Imbalance in dopamine production and metabolism has been implicated in psychiatric and neurodegenerative diseases and disorders such as schizophrenia, depression, addiction, and Parkinson's disease. Schizophrenia is a severe and chronic mental illness (or a group of illnesses), associated with high prevalence (0.5-1% of the population suffers from this condition). Positive symptoms of the disorder such as hallucinations and paranoia are responsive to neuroleptics in most of the patients. Negative symptoms including emotional withdrawal, motor retardation, and cognitive impairments such as working memory deficits, are usually not affected by neuroleptics.

Schizophrenia is associated with disruption of neurotransmission in specific brain regions in humans and in animal models with several schizophrenic phenotypes. Functional imaging studies showed that the cognitive deficits in schizophrenia might arise from altered prefrontal cortex function. Indirect evidence supports the hypothesis that a deficit in prefrontal dopamine function might contribute to prefrontal impairment in schizophrenia. The only index of prefrontal dopamine transmission currently quantifiable in vivo is D₁ receptor availability by PET imaging. Results of studies using radiotracers for D₁ are in agreement with the hypothesis that a deficit in prefrontal dopamine activity at D₁ receptors might contribute to the cognitive problems presented by patients with schizophrenia. Clinical studies have suggested a relationship between low cerebro-spinal fluid homovanillic acid (a dopamine metabolite) and poor performance in tasks involving working memory but not in nonprefrontal task. However, direct evidence of brain regions in which dopamine synthesis or metabolism are altered is not available.

Several lines of evidence suggest that schizophrenia might also be associated with a persistent dysfunction of glutamate transmission involving NMDA receptors. Noncompetitive NMDA antagonists such as phencyclidine or ketamine, induce both positive and negative symptoms in healthy subjects and patients with schizophrenia. Unmediated patients with schizophrenia are more sensitive than normal subjects to the effects of NMDA antagonists. However, direct evidence for NMDA dysfunction or altered glutamate synthesis and metabolism in schizophrenia is still lacking.

Typical neuroleptics block the dopamine receptor 2 (D₂). Their success in ameliorating psychotic symptoms first led to the dopaminergic hypothesis of schizophrenia. While the known biological processes that are involved in this therapy are fairly fast (receptor binding), typically, there is a several weeks time lag between the onset of treatment and the start of therapeutic benefits. The reason for this time lag is not known.

Similarly to the cases of SSRIs and AChE inhibitors, neuroleptics have side effects, not all patients respond to a specific treatment, and many times patients have to switch between drug regimes until the best drug for them is found by educated trial and error. This phase of trial and error could last several weeks to several months because there is no test for determining the direct drug action and efficacy in the individual's brain.

The various modulatory systems of the brain, the serotonergic, dopaminergic, cholinergic and adrenergic systems, do not function independently of each other but rather interact at several levels. Specifically the distribution of the serotonergic system overlaps with and interacts with the noradrenergic system. Moreover, receptors for the two amines coexist on the same neurons, and there is cross talk between second messengers activated by these transmitters. The balance between the neuromodulatory systems in the human brain is important for brain function, whereas an imbalance has been implicated in several diseases including schizophrenia, depression, PD, and AD.

In summary, brain metabolism, specifically neuromodulator metabolism (serotonin, dopamine, and acetylcholine) has been implicated in the regulation of movement, thought, volition, and mood. Most of the psychiatric drugs and neuroprotective drugs are targeted toward at least one aspect of neuromodulator metabolism and action. However, most of these processes, including neuromodulators' metabolism, can not be directly detected in a non-invasive manner.

The synthesis of Nitrous Oxide (NO) is important for the regulation of blood flow. Changes in blood flow and NO production have been shown to be associated with numerous psychiatric and neurologic conditions as well as with kidney, liver, and muscle function, and atherosclerosis. It is known in the art that NO is produced through the conversion of arginine to citrulline. However, this reaction, as well as other aspects of NO metabolism) have not been directly observed in the living human brain or body in a non-invasive manner.

N-acetylaspartate (NAA) is another neurochemical that has been implicated in psychiatric and neurodegenerative diseases. There is a strong correlation between low NAA levels (as determined non-invasively by localized magnetic resonance spectroscopy) and various neurodegenerative processes. In schizophrenia, ¹H-MRS studies showed unequivocally that the prefrontal NAA concentration or the NAA to creatine ratio was decreased, even in neuroleptic naïve patients. However, it is still not clear whether a decrease in NAA levels is a cause or effect of neurodegeneration and how well the total NAA level can be used in the diagnosis of a neurodegenerative state in the individual's brain. The metabolic pathways of NAA in the human brain have not been explored in a non-invasive manner yet.

Clinical and in vivo studies in animals use determination of neuromodulator metabolites in body fluids rather than in the active brain region. Despite the numerous processes that are involved in metabolite secretion from the brain and retention in the body fluids, a relationship between metabolism and specific brain functions had been observed. For example, low cerebro-spinal fluid homovanillic acid (a dopamine metabolite) was found to correlate with poor performance in tasks involving working memory but not in nonprefrontal tasks. In animal models, using invasive methods, numerous studies have shown a relationship between altered metabolism in specific brain regions and behavior. An overwhelming effort has been directed at developing cerebrospinal fluid biomarkers or blood biomarkers for early diagnosis of psychiatric and neurological conditions such as Alzheimer's disease and bipolar depression. Thus far, such a biomarker that will enable a differential diagnosis and a clear treatment indication has not been found. Therefore, the ability to monitor neuromodulator metabolism and other metabolic processes in specific brain regions, in a non-invasive manner, is important for characterizing the control on brain function, making differential diagnoses, and guiding and monitoring treatment.

The various levels anesthesia are associated with varying electrical brain activity waves as well as variation in neuromodulatory activity and balance. Therefore, the ability to monitor neuromodulator metabolism in specific brain regions, in a non-invasive manner, may provide an objective biomarker to the level of anesthesia.

Determination of the degree of comatose states is even vaguer than that of the level of anesthesia. Therefore, the ability to monitor neuromodulator metabolism in specific brain regions, in a non-invasive manner, may provide an objective biomarker for characterizing (and potentially treating) this condition(s).

Neurostimulation in general and deep brain stimulation specifically, show promising new tools for controlling erroneous brain function. However, the evaluation of the need for this treatment and the localization of such electrodes within the brain are lacking objective biomarker for the location of the dysfunctional neuromodulatory area within the brain. Therefore, the ability to monitor neuromodulator metabolism in specific brain regions, in a non-invasive manner, may provide objective and standardized biomarkers for this treatment approach.

In the cases of trauma and stroke it is important to determine the extent of the affected penumbra. In both cases, changes in neuromodulators follow the neuronal damage, but in a larger area compared to the original damage. It is known in the art that the extent of this penumbra has a strong predictive value and guides treatment options. An extensive effort has been devoted to developing non-invasive means for visualizing the affected penumbra. Therefore, the ability to monitor neuromodulator metabolism in specific brain regions, in a non-invasive manner, may provide objective and standardized biomarkers to aid in stratifying treatment.

Currently, the most widely used methods for imaging of the human brain are computerized tomography (CT), magnetic resonance imaging (MRI), and positron emission tomography (PET). While CT provides mainly anatomical information, functional MRI (fMRI) and PET are able to provide added information on brain activation. fMRI makes use of MRI to measure the hemodynamic signals related to the changes in cerebral blood flow, volume, and oxygenation. PET is a method for imaging that uses tracers that emit positrons. The tracer is introduced into the subject's blood and then its concentration is measured using the emitted positrons. PET is used for measuring cerebral blood flow and tracer uptake and retention. Both fMRI and PET rely on activation induced changes in blood flow, blood volume, oxygen consumption, and glucose consumption. However, the relationship between these changes and neuronal activity remains unclear, especially in the case of neuromodulation. Neuromodulation is not excitatory or inhibitory in the neurotransmitter sense (for example, a neuromodulator may inhibit an inhibitory message), therefore, areas of neuromodulator synthesis, metabolism, and release, may not overlap with areas of activation identified by fMRI and PET. Moreover, neuromodulatory neurons are able to secrete more than one type of neurotransmitter (for example dopamine and glutamate). PET imaging also makes use of radioactively labeled ligands for neuromodulator receptors, transporters, and other brain macromolecules, thus enabling visualization of the levels of these macromolecules in a non-invasive, albeit radioactive manner. Therefore, visualization of neuromodulator metabolism and its correlation with brain activation, as visualized by fMRI and PET, may aid in understanding of the network activity of in the brain, characterizing the neuromodulatory system activity of the individual, making a differential diagnosis, and monitoring treatment.

Brain function is also investigated by several other means such as electrophysiology, electroencephalography (EEG), and single-photon emission computed tomography (SPECT): During an electrophysiological investigation, electrodes or an electrode array are being placed at specific locations within the brain by an invasive procedure, and the electrical behavior of the brain tissue is measured at that location. EEG and computerized EEG are noninvasive, diagnostic techniques that record the electrical impulses produced by brain cell activity and reveal characteristic brain wave patterns that may assist in the diagnosis of particular neurologic conditions. SPECT is a special type of computed tomography (CT) scan in which a small amount of a radioactive drug is injected into a vein and a scanner is used to make detailed images of areas inside the brain where the radioactive material is present.

Magnetic resonance spectroscopy (MRS) is currently the only method that enables direct non-invasive detection of metabolism in specific regions of the living brain. MRS utilizes the differences in the chemical surrounding of individual nuclei in molecules, which results in differences in resonance frequencies, to identify specific molecules. Localized MRS utilizes sequences of radio-frequency pulses and pulsed field gradients to obtain spectra of specific regions in the brain. These spectra can be interpreted to provide information on the content of endogenous compounds and exogenous agents. Carbon-13 brain MRS has been used in animals and humans to monitor the synthesis of glutamate, glutamine, aspartate, GABA, and lactate. The ¹³C-MRS methodology was recently applied in rat brain slices and enabled direct detection of acetylcholine synthesis, demonstrating the use ¹³C-MRS for direct non-invasive detection of neuromodulatory activity. However, currently, the low (micro-molar range) concentration of neuromodulators prevents in vivo detection by MRS at high resolution. To enable high resolution ¹³C-MRS studies of neuromodulation in the intact brain, an improvement of several orders of magnitude in the signal-to-noise ratio is needed. Such an improvement has been achieved by hyperpolarization methods which are described below.

The underlying principle of MRI and MRS is based on the interaction of atomic nuclei with an external magnetic field. A fundamental property of the atomic nucleus is the nuclear spin, described by the spin quantum number I. Many atomic nuclei have a non-zero spin quantum number and can be studied with nuclear magnetic resonance (NMR). However, the clinical use of MRI has to date been restricted to ¹H, for reasons of sensitivity. Not only does ¹H have a higher sensitivity than any other nucleus in endogenous substances; it is also abundant in very high concentration (about 80 M) in biological tissues.

Nuclei with spin quantum number I=½ (such as ¹H, ¹³C, and ¹⁵N) can be oriented in two possible directions: parallel (“spin up”) or anti-parallel (“spin down”) to the external magnetic field. The net magnetization per unit volume, and thus the available NMR signal, is proportional to the population difference between the two states. If the two populations are equal, their magnetic moments cancel, resulting in zero macroscopic magnetization, and thus no NMR signal. However, under thermal equilibrium conditions, slightly higher energy is associated with the “spin down” direction, and the number of such spins will thus be slightly smaller than the number of spins in the “spin up” state.

The polarization (P) of any given nucleus can be defined as P═CB₀/T, where C is a nucleus specific constant, B₀ is the magnetic field strength, and T is the absolute temperature. The thermal equilibrium polarization is very low: even at a magnetic field of 1.5 T it is only 5×10⁻⁶ for ¹H, and 1×10⁻⁶ for ¹³C (at body temperature). In other words, only about one of a million nuclei contributes to the measured NMR signal in a standard clinical MRI scanner. The polarization, and thereby the strength of the NMR signal, increases proportionally with the magnetic field, which has been the motivation for developing higher field MRI systems.

A conceptually different method to increase the polarization is to create an artificial, non-equilibrium distribution of the nuclei: the “hyperpolarized” state, where the population difference (“spin up”-“spin down”) is increased by several orders of magnitudes compared with the thermal equilibrium. The hyperpolarized state can be created in vivo by means of dynamic nuclear polarization (DNP) techniques, such as the Overhauser effect, in combination with a suitable contrast agent. Alternatively, it is known in the art that the hyperpolarized state of an imaging agent can be created by an external device, followed by rapid administration of the agent to the subject to be imaged. It is known in the art that it is possible to hyperpolarize a wide range of organic molecules containing ¹³C or ¹⁵N, by either dynamic nuclear polarization (DNP) or parahydrogen-induced polarization (PHIP), and reach up to five orders of magnitude increase in the signal of ¹³C-MRS of the agent in liquid state. The present invention describes neurochemical agents for use at thermal equilibrium or at a hyperpolarized state created by such external hyperpolarization methods.

Using the present invention, the DNP and PHIP methods are harnessed for non-invasive studies of neuromodulation and neurochemistry in the intact brain and body, using specific neurochemical and biochemical agents that are hyperpolarized ex-vivo.

Magnetic resonance imaging and spectroscopy (MRI/MRS) has become particularly attractive to physicians as a diagnostic technique because it is non-invasive and does not involve exposing the patient under study to potentially harmful ionizing radiation. In order to achieve effective contrast between MR images of the different tissue types in a subject, it has long been known in the art to administer to the subject MR contrast agents (e.g. paramagnetic metal species) that affect relaxation times of the MR imaging nuclei in the regions in which they are administered or at which they aggregate. The same principle has also been utilized in metabolic studies where ¹³C-labeled agents are administered to enhance the ability to detect that particular agent and its metabolic fates. Contrast enhancement has also been achieved by utilizing the “Overhauser effect” in which an Electron Spin Resonance (ESR) transition in an administered paramagnetic species (hereinafter an OMRI contrast agent) is coupled to the nuclear spin system of the imaging nuclei. The Overhauser effect (also known as dynamic nuclear polarization) can significantly increase the polarization of selected nuclei and thereby amplify the MR signal intensity by a factor of a hundred or more allowing OMRI images to be generated rapidly and with relatively low primary magnetic fields. In is known in the art that radicals can be used as OMRI contrast agents and effect polarization of imaging nuclei in vivo and ex-vivo.

It is known in the art that there are techniques which involve ex vivo polarization of agents containing MR imaging nuclei, prior to administration and MR signal measurement. Such techniques may involve the use of polarizing agents, for example conventional OMRI contrast agents, hyperpolarized gases, or hydrogenation catalysts to achieve ex vivo polarization of administrable MR imaging nuclei. By polarizing agent is meant any agent suitable for performing ex vivo polarization of an MR imaging or spectroscopic agent.

The ex vivo method has the advantage that it is possible to avoid administering the whole of, or substantially the whole of, the polarizing agent to the sample under investigation, whilst still achieving the desired polarization. Thus the administration of the spectroscopic or imaging agent is less constrained by physiological factors such as the constraints imposed by the administrability, biodegradability, and toxicity of OMRI, DNP, and PHIP contrast agents and catalysts in in vivo techniques.

DNP may be attained by three possible mechanisms: (1) the Overhauser effect, (2) the solid effect and (3) thermal mixing effect. The Overhauser effect is a relaxation driven process that occurs when the electron-nucleus interaction is time-dependent (due to thermal motion or relaxation effects) on the time scale of the inverse electron Larmor frequency or shorter. Electron-nuclear cross-relaxation results in an exchange of energy with the lattice giving rise to an enhanced nuclear polarization. The overall enhancement depends on the relative strength of the scalar and dipolar electron-nuclear interaction and the microwave power. In the solid effect, the electron spin system is irradiated at a frequency that corresponds to the sum or the difference of the electronic and nuclear Larmor frequencies. The nuclear Zeeman reservoir absorbs or emits the energy difference and its spin temperature is modified, resulting in an enhanced nuclear polarization. The efficiency depends on the transition probabilities of otherwise forbidden transitions that are allowed due to the mixing of nuclear states by non-secular terms of the electron-nuclear dipolar interaction. Thermal mixing arises when the electron-electron dipolar reservoir establishes thermal contact with the nuclear Zeeman reservoirs. This takes place when the characteristic electronic resonance line width is of the order of the nuclear Larmor frequency. Electron-electron cross relaxation between spins with difference in energy equal to the nuclear Zeeman energy is absorbed or emitted by the electronic dipolar reservoir, changing its spin temperature and the nuclear polarization is enhanced. For thermal mixing both the forbidden and the allowed transitions can be involved.

It is known in the art that where the polarizing agent is an OMRI contrast agent, the polarization may be carried out by using a first magnet for providing the polarizing magnetic field and a second magnet for providing the primary magnetic field for MR imaging. In the first magnet, a dielectric resonator is used in the DNP process. Simplistically, it is known in the art that DNP requires a volume with a fairly strong high frequency magnetic field and an accompanying electric field which is made as small as possible. A dielectric resonator is used to provide a preferred field arrangement in which the magnetic field lines are shaped like a straw in a sheaf of corn with an electric field forming circles like the thread binding the sheaf. The composition to be polarized is placed inside the resonator which is itself placed inside a metal box with a clearance typically of the order of the size of the resonator, and is excited to the desired resonance with a coupling loop or the like. An alternative to the dielectric resonator is a resonant cavity. One simple and efficient resonant cavity is a metal box, such as a cylindrical metal box. A suitable mode is the one known as TM1,1,0 which produces a perpendicular magnetic field on the axis of the cavity.

In solids, it is preferred to effect dynamic nuclear polarization by irradiating an electron spin at low temperature and high field. It is known in the art that the electron spin sources could be free radicals that are known in the art such as: 4-amino TEMPO, TEMPO, and complexes of Cr. Preferably of course a chosen OMRI contrast agent will exhibit a long half-life (preferably at least one hour), long relaxation times (T₁ and T₂), high relativity and a small number of ESR transition lines. Thus the paramagnetic oxygen-based, sulphur-based or carbon-based organic free radicals or magnetic particles, referred to in WO-A-68/10419, WO-A-90/00904, WO-A-91/12024, WO-A-93/02711 or WO-A-96/39367 would be suitable OMRI contrast agents. A particularly preferred characteristic of a chosen OMRI contrast agent is that it exhibits low inherent ESR linewidths, preferably less than 500 mG, particularly preferably less than 400 mG, especially preferably less than 150 mG. Generally speaking, organic free radicals such as triarylmethyl and nitroxide radicals provide the most likely source of such desirably low linewidths e.g. those described in WO-A-88/10419, WO-A-90/00904, WO-A-91/12024, WO-A-93/02711 or WO-A-96/39367.

After the polarization and prior to administration of the hyperpolarized spectroscopic or imaging agent into the sample, it is desirable to remove substantially the whole of the OMRI contrast agent from the composition (or at least to reduce it to physiologically tolerable levels) as rapidly as possible. Many physical and chemical separation or extraction techniques are known in the art and may be employed to effect rapid and efficient separation of the OMRI contrast agent and the spectroscopic or imaging agent. Clearly the more preferred separation techniques are those which can be applied rapidly and particularly those which allow separation in less than one second. In this respect, magnetic particles (e.g. superparamagnetic particles) may be advantageously used as the OMRI contrast agent as it will be possible to make use of the inherent magnetic properties of the particles to achieve rapid separation by known techniques. Similarly, where the OMRI contrast agent or the particle is bound to a solid bead, it may be conveniently separated from the liquid (i.e. if the solid bead is magnetic by an appropriately applied magnetic field).

For ease of separation of the OMRI contrast agent and the spectroscopic or imaging agent, it is particularly preferred that the combination of the two be a heterogeneous system, e.g. a two phase liquid, a solid in liquid suspension or a relatively high surface area solid substrate within a liquid, e.g. a solid in the form of beads fibers or sheets disposed within a liquid phase spectroscopic or imaging agent. In all cases, the diffusion distance between the spectroscopic or imaging agent and the OMRI contrast agent must be small enough to achieve an effective Overhauser enhancement. Certain OMRI contrast agents are inherently particular in nature, e.g. the paramagnetic particles and superparamagnetic agents referred to above. Others may be immobilized on, absorbed in or coupled to a solid substrate or support (e.g. an organic polymer or inorganic matrix such as a zeolite or a silicon material) by conventional means. Strong covalent binding between OMRI contrast agent and solid substrate or support will, in general, limit the effectiveness of the agent in achieving the desired Overhauser effect and so it is preferred that the binding, if any, between the OMRI contrast agent and the solid support or substrate is weak so that the OMRI contrast agent is still capable of free rotation. The OMRI contrast agent may be bound to a water insoluble substrate/support prior to the polarization or the OMRI contrast agent may be attached/bound to the substrate/support after polarization. The OMRI contrast agent may then be separated from the spectroscopic or imaging agent e.g. by filtration before administration. The OMRI contrast agent may also be bound to a water soluble macromolecule and the OMRI contrast agent-macromolecule may be separated from the spectroscopic or imaging agent before administration.

Where the combination of an OMRI contrast agent and a spectroscopic or imaging agent is a heterogeneous system, it will be possible to use the different physical properties of the phases to carry out separation by conventional techniques. For example, where one phase is aqueous and the other non-aqueous (solid or liquid) it may be possible to simply decant one phase from the other. Alternatively, where the OMRI contrast agent is a solid or solid substrate (e.g. a bead) suspended in a liquid spectroscopic of imaging agent the solid may be separated from the liquid by conventional means e.g. filtration, gravimetric, chromatographic or centrifugal means. The spectroscopic or imaging agent may also be in a solid (e.g. frozen) state during polarization and in close contact with a solid OMRI contrast agent. After polarization it may be dissolved in heated water or saline or melted and removed or separated from the OMRI contrast agent where the latter may be toxic and cannot be administered.

One separation technique makes use of a cation exchange polymer and a cationic OMRI contrast agent, e.g. a triarylmethyl radical carrying pendant carboxylate groups. Alternatively acidifying the solution to around pH 4 may cause the OMRI contrast agent to precipitate out. Separation may then be carried out for example by filtration followed by neutralization. An alternative technique involves adding ions which causes precipitation of ionic OMRI agents which may then be filtered out.

Certain OMRI contrast agents, such as the triarylmethyl radical, may have an affinity for proteins. Thus, after polarization, a composition containing an OMRI contrast agent with a protein affinity may be passed through or over a protein in a form which exposes a large surface area to the agent e.g., in particulate or surface bound form. In this way, binding of the OMRI contrast agent to the protein enables it to be removed from the composition. Other possible electron spin sources known in the art include particles exhibiting the magnetic properties of paramagnetism, superparamagnetism, ferromagnetism or ferromagnetism may also be useful OMRI contrast agents, as may be other particles having associated free electrons. Superparamagnatic nanoparticles (e.g. iron or iron oxide nanoparticles) may be particularly useful. Magnetic particles have the advantages over organic free radicals of high stability and a strong electronic/nuclear spin coupling (i.e. high relaxivity) leading to greater Overhauser enhancement factors.

PHIP may be attained by parahydrogen hydrogenation of a double or triple carbon-carbon bond in a molecule that contains carbon-13 (preferably in a position that is close to the unsaturated bond). Parahydrogen is the singlet state of the nuclear spins of dihydrogen. This is one of the four possible spin isomers of the dihydrogen molecule ψ_{P}=1/√2(|αβ)-|ββ>) which has the lowest energy. This spin isomer dominates at temperatures below 77 K, the temperature of liquid nitrogen. A transfer of the parahydrogen molecule as a unit onto the substrate is a requisite for the PHIP effect to take place. A ¹³C-labeled molecule serves to break the symmetry and the increased spin order effect can be detected using proton spectroscopy by the appearance of strong antiphase signals. The spin order of the parahydrogen molecule is then converted to nuclear polarization of the ¹³C nucleus, via a nonadiabatic field cycling scheme. This field cycling includes a sudden decrease in the external magnetic field (≈3×10⁻⁸ T in 1 ms) and a gradual increase of the field back to the ambient earth's magnetic field (≈10⁻⁴ T). This field cycling results in a rearrangement of the populations of the original eigenstates of the Hamiltonian so that the system now displays an NMR spectrum where the allowed transitions are predominantly in phase, corresponding to a substantial polarization. It is known in the art that using the PHIP method it is possible to achieve up to five orders of magnitude increase in the ¹³C-MRS signal of ¹³C-labeled agents and naturally abundant ¹³C nuclei in non-enriched compounds.

After the polarization and prior to administration of the PHIP hyperpolarized spectroscopic or imaging agent into the sample, it is desirable to remove substantially the whole of the hydrogenation catalyst from the composition (or at least to reduce it to physiologically tolerable levels) as rapidly as possible. Many physical and chemical separation or extraction techniques are known in the art and may be employed to effect rapid and efficient separation of the catalyst and the spectroscopic or imaging agent. Clearly the more preferred separation techniques are those which can be employed rapidly and particularly those which allow separation in less than one second.

For ease of separation of the hydrogenation catalyst and the spectroscopic or imaging agent, it is particularly preferred that the combination of the two be a heterogeneous system, e.g. a two phase liquid, nanoparticles in water (where water molecules surrounding nanoparticles form water with organic solvent capability), a solid in liquid suspension or a relatively high surface area solid substrate within a liquid, e.g. a solid in the form of beads fibers or sheets disposed within a liquid phase spectroscopic or imaging agent. Hydrogenation catalysts may be immobilized on, absorbed in or coupled to a solid substrate or support (e.g. an organic polymer or inorganic matrix such as a zeolite or a silicon material) by conventional means. The hydrogenation catalyst can be separated from the spectroscopic or imaging agent e.g. by filtration before administration. The hydrogenation catalyst may also be bound to a water soluble macromolecule and the hydrogenation catalyst-macromolecule may be separated from the spectroscopic or imaging agent before administration.

Where the combination of a hydrogenation catalyst and a spectroscopic or imaging agent is a heterogeneous system, it will be possible to use the different physical properties of the phases to carry out separation by conventional techniques. For example, where one phase is aqueous and the other non-aqueous (solid or liquid) it may be possible to simply decant one phase from the other. Alternatively, where the hydrogenation catalyst is a solid or solid substrate (e.g. a bead) suspended in a liquid spectroscopic or imaging agent the solid may be separated from the liquid by conventional means e.g. filtration, gravimetric, chromatographic or centrifugal means.

### SUMMARY OF THE INVENTION

The present invention provides neurochemical and biochemical agents, device, and methods for direct, non-invasive, quantification of neuronal function, brain function, and general biochemistry. The temporal and spatial distribution of the neurochemical and biochemical metabolism is quantified and provides markers of specific brain activity, psychiatric and neurodegenerative diseases and disorders, and therapeutic action and efficacy. Said method comprising the step of ex vivo polarization of the neurochemical agent, administration of this hyper-polarized agent to the human or the animal body or brain, and monitoring of the distribution of this agent and its metabolic fates in the brain by magnetic resonance spectroscopy and imaging. Said device comprised of a system for detection and analysis of both hyper-polarized and thermal equilibrium neurochemical signals, quantification of specific metabolites, and presentation of the metabolic results fused with the anatomic and functional images of the brain (or body) with operating modules of magnetic resonance scanner, polarizer, and software for image and spectra analysis.

It has now been found that in vivo methods of magnetic resonance imaging and spectroscopy may be improved by using ex-vivo polarized MR agents comprising nuclei capable of emitting magnetic resonance signals in a uniform magnetic field (e.g. MR nuclei such as ¹³C, ¹⁵N, or ¹⁹F nuclei) and capable of exhibiting a long T₁ relaxation time, preferably additionally a long T₂ relaxation time, ability to cross the blood brain barrier, and optionally, an ability to be metabolized in the brain or body. Such agents will be referred to hereinafter as “high T₁ neurochemical agents” or HTNC agents. Typically the HTNC agent molecules will contain MR imaging/spectroscopic nuclei in an amount greater than the natural abundance of said nuclei in said molecules (i.e. the agent will be enriched with said nuclei).

It is in the scope of the present invention to provide a system for detection and analysis of hyper-polarized and thermal equilibrium signals, quantification of specific neurochemical and biochemical metabolites, and presentation of the metabolic results fused with the anatomic and functional images of the brain (or body) comprising operating modules of magnetic resonance scanner, polarizer, and software for image and spectra analysis.

It is also in the scope of the present invention to provide a method for detecting the spatial and temporal distribution of neurochemicals and their metabolic/catabolic products within the human brain or body, comprising at least one step of ex vivo polarization of at least one neurochemical agent, administrating said hyper-polarized agent to a human's or animal's body or brain, monitoring the distribution of said agent or agents and its metabolic successors in the brain by magnetic resonance spectroscopy and imaging.
a) Said method may comprise steps selected inter alia from: subjecting a high T₁ neurochemical (HTNC) agent to ex vivo polarization and where this is carried out by means of a polarizing agent or catalyst and polarization apparatus, optionally separating the whole, or a portion of said polarizing agent or catalyst from said HTNC agent;
b) administering said HTNC agent to the human or non-human animal body or brain;
c) exposing said body or brain to a radiation of a frequency selected to excite nuclear spin transitions in selected nuclei;
d) detecting magnetic resonance signals from said body or brain;
e) optionally, generating image, metabolic data, enzyme kinetics data, diffusion data, relaxation data, or physiological data from said detected signals;
f) optionally, use of the data obtained in step (e) to aid in quantifying neuronal and brain function;
g) optionally, use of the data obtained in step (f) to diagnose diseases and disorders of the body or brain;
h) optionally, use of the data obtained in steps (f) and (g) to monitor action of and response to therapy aimed at alleviating or curing psychiatric, neurodegenerative, and neurological diseases and disorders;
i) optionally, use of the data obtained in step (f) to affirm drug activity in situ and determine drug efficacy;
j) optionally, use of data obtained in step (f) for strategic planning of the location of neurostimulation electrodes;
k) optionally, use of data obtained in step (f) for strategic planning of the location of slow-release or controlled release devices within the body or brain;
l) optionally, use of data obtained in step (f) for characterization of masses, tumors, cysts, blood vessel abnormalities, and internal organ function;
m) optionally, use of the data obtained in step (f) for evaluation and determination of the level of anesthesia, comatose states, and the brain regions affected by stroke or trauma and their penumbra;
wherein said HTNC agent is a solid or liquid HTNC agent comprising nuclei selected from the group consisting of ¹H, ¹³C, ¹⁵N, ¹⁹F and ³¹P nuclei and wherein said solid HTNC agent is dissolved in an administrable media prior to administration to said sample.

It is also in the scope of the present invention wherein said HTNC agent has a T₁ value at a field strength of 0.01-5 T and a temperature in the range 20-40° C. of at least 2 seconds.

It is also in the scope of the present invention wherein said HTNC agent has a T₁ value at field strength of 0.01-5 T and a temperature in the range 20-40° C. of at least 5 seconds.

It is also in the scope of the present invention wherein said HTNC agent has a T₁ value at field strength of 0.01-5 T and a temperature in the range 20-40° C. of at least 10 seconds.

It is also in the scope of the present invention wherein said HTNC agent has a T₁ value at field strength of 0.01-5 T and a temperature in the range 20-40° C. of at least 30 seconds.

It is also in the scope of the present invention wherein said HTNC agent has a T₁ value at field strength of 0.01-5 T and a temperature in the range 20-40° C. of at least 70 seconds.

It is also in the scope of the present invention wherein said HTNC agent has a T₁ value at field strength of 0.01-5 T and a temperature in the range 20-40° C. of at least 100 seconds.

It is also in the scope of the present invention wherein said HTNC agent has a T₁ value at field strength of 0.01-5 T and a temperature in the range 20-40° C. of at least 200 seconds.

It is also in the scope of the present invention wherein said HTNC agent has a T₁ value at field strength of 0.01-5 T and a temperature in the range 20-40° C. of at least 300 seconds.

It is also in the scope of the present invention wherein said HTNC agent comprising ¹³C nuclei.

It is also in the scope of the present invention wherein said HTNC agent has ¹³C at one particular position in its molecular structure in an amount above 1%.

It is also in the scope of the present invention wherein said HTNC agent has ¹³C at one particular position in its molecular structure in an amount above 5%.

It is also in the scope of the present invention wherein said HTNC agent has ¹³C at one particular position in its molecular structure in an amount above 10%.

It is also in the scope of the present invention wherein said HTNC agent has ¹³C at one particular position in its molecular structure in an amount above 25%.

It is also in the scope of the present invention wherein said HTNC agent has ¹³C at one particular position in its molecular structure in an amount above 50%.

It is also in the scope of the present invention wherein said HTNC agent has ¹³C at one particular position in its molecular structure in an amount above 99%.

It is also in the scope of the present invention wherein said high HTNC agent is ¹³C enriched at one or more carbon positions.

It is also in the scope of the present invention wherein said high HTNC agent is deuterium labeled at one or more proton positions.

It is also in the scope of the present invention wherein said deuterium label is adjacent a ¹³C nucleus.

It is also in the scope of the present invention wherein said HTNC agent contains ¹⁹F nuclei.

It is also in the scope of the present invention wherein said HTNC agent contains ¹⁵N nuclei.

It is also in the scope of the present invention wherein said HTNC agent has ¹⁵N at one particular position in its molecular structure in an amount above 1%.

It is also in the scope of the present invention wherein said HTNC agent has ¹⁵N at one particular position in its molecular structure in an amount above 5%.

It is also in the scope of the present invention wherein said HTNC agent has ¹⁵N at one particular position in its molecular structure in an amount above 10%.

It is also in the scope of the present invention wherein said HTNC agent has ¹⁵N at one particular position in its molecular structure in an amount above 25%.

It is also in the scope of the present invention wherein said HTNC agent has 15N at one particular position in its molecular structure in an amount above 50%.

It is also in the scope of the present invention wherein said HTNC agent has ¹⁵N at one particular position in its molecular structure in an amount above 99%.

It is also in the scope of the present invention wherein said HTNC agent is enriched with ¹⁵N at one or more nitrogen positions.

It is also in the scope of the present invention wherein said polarizing agent or catalyst is used in liquid or solid form.

It is also in the scope of the present invention wherein the use of the said polarization agent or hydrogenation catalyst and polarization apparatus increased the polarization of the HTNC agent two fold (compared to the polarization level at identical physical and chemical conditions without the use of said polarization agent or catalyst and polarization apparatus).

It is also in the scope of the present invention wherein the use of the said polarization agent and polarization apparatus increased the polarization of the HTNC agent by 10 fold.

It is also in the scope of the present invention wherein the use of the said polarization agent or hydrogenation catalyst and polarization apparatus increased the polarization of the HTNC agent by 50 fold.

It is also in the scope of the present invention wherein the use of the said polarization agent or hydrogenation catalyst and polarization apparatus increased the polarization of the HTNC agent by 100 fold.

It is also in the scope of the present invention wherein the use of the said polarization agent or hydrogenation catalyst and polarization apparatus increased the polarization of the HTNC agent by 500 fold.

It is also in the scope of the present invention wherein the use of the said polarization agent or hydrogenation catalyst and polarization apparatus increased the polarization of the HTNC agent by 1,000 fold.

It is also in the scope of the present invention wherein the use of the said polarization agent or hydrogenation catalyst and polarization apparatus increased the polarization of the HTNC agent by 5,000 fold.

It is also in the scope of the present invention wherein the use of the said polarization agent and or hydrogenation catalyst polarization apparatus increased the polarization of the HTNC agent by 10,000 fold.

It is also in the scope of the present invention wherein the use of the said polarization agent and or hydrogenation catalyst polarization apparatus increased the polarization of the HTNC agent by 50,000 fold.

It is also in the scope of the present invention wherein the use of the said polarization agent or hydrogenation catalyst and polarization apparatus increased the polarization of the HTNC agent by 100,000 fold.

It is also in the scope of the present invention wherein the use of the said polarization agent or hydrogenation catalyst and polarization apparatus increased the polarization of the HTNC agent by 500,000 fold.

Thus viewed from one aspect the present invention provides a method of magnetic resonance metabolic investigation of a human or non-human animal body or brain, said method comprising steps selected in a non-limiting manner from:
i. subjecting a HTNC agent to ex vivo polarization;
ii. optionally exposing the HTNC agent to a uniform magnetic field (e.g. the primary field B₀ of the imaging apparatus of a weaker field e.g. 1 G or more);
iii. where step (i) is carried out by means of a polarizing agent or hydrogenation catalyst, optionally separating the whole, substantially the whole, or a portion of said polarizing agent or hydrogenation catalyst from said HTNC agent;
iv. administering said HTNC agent to said human or animal body or brain;
v. exposing said body or brain to a second radiation of a frequency selected to excite nuclear spin transitions in selected nuclei e.g. the MR spectroscopic or imaging nuclei of the HTNC agent;
vi. detecting magnetic resonance signals from said body or brain; and
vii. optionally, generating image, metabolic data, enzyme kinetics data, diffusion data, relaxation data, or physiological data from said detected signals;
viii. optionally, use of the data obtained in vii) to aid in quantifying neuronal function;
ix. optionally, use of the data obtained in viii) to diagnose diseases and disorders of the body or brain;
x. optionally, use of the data obtained in vii) and viii) to monitor action of and response to therapy aimed at alleviating or curing psychiatric, neurodegenerative, and neurological diseases and disorders;
xi. optionally, use of the data obtained in viii) to affirm drug activity in situ and determine drug efficacy;
xii. optionally, use of data obtained in viii) for strategic planning of the location of neurostimulation electrodes;
xiii. optionally, use of data obtained in viii) for strategic planning of the location of slow-release or controlled release devices within the brain;
xiv. optionally, use of data obtained in step (f) for characterization of masses, tumors, cysts, blood vessel abnormalities, and internal organ function;
xv. optionally, use of the data obtained in step (f) for evaluation and determination of the level of anesthesia, comatose states, and the brain regions affected by stroke or trauma and their penumbra;

Thus the invention involves the sequential steps of ex vivo polarization of a HTNC agent comprising nuclei capable of exhibiting a long T₁ relaxation time, administration of the polarized HTNC agent (preferably in the absence of a portion of, more preferably substantially the whole of, any polarizing agent or catalyst), and conventional in vivo MR signal generation and measurement. The MR signals obtained in this way may be converted by conventional manipulations into 2-, 3- or 4-dimensional data including metabolic, kinetic, diffusion, relaxation, and physiological data.

Viewed from a further aspect the present invention provides a composition comprising a polarized ¹³C, ¹⁵N, ²H, or ¹⁹F enriched compound together with one or more physiologically acceptable carriers, excipients, protection, or function modulation agents. Viewed from a further aspect the present invention provides a contrast medium comprising a polarized HTNC agent being enriched with ¹³C nuclei, ¹⁵N, ²H, or ¹⁹F having a T₁ relaxation time of about 2 s or more in solution at magnetic fields of about 0.005 to about 10 T, together with one or more physiologically acceptable carriers, excipients, protection, or function modulation agents.

The HTNC agents include molecules of metabolic potential such as: choline, betaine, acetylcholine, acetate, aspartate, N-acetylaspartate, creatine, L-tyrosine, L-DOPA, dopamine, norepinephrine, epinephrine, vanillylmandelic acid (VMA), homovanillic acid (HVA), 3-O-methyldopamine, 3-O-methylnorepinephrine, 3-O-methylepinephrine, dopaquinone, L-tryptophan, 5-hydroxy-tryptophan, serotonin, 5-hydroxyindole acetaldehyde, 5-hydroxyindole acetic acid, melatonin, glutamate, arginine, citrulline, N-acetylcitrulline, argininosuccinate, kynurenic acid (KYNA), 7-chlorokynurenic acid (7-Cl—KYNA), kynurenine, and 4-chlorokynurenine, and pharmacologically acceptable salts thereof, and combinations of any of the foregoing;

The HTNC agents also include molecules that are currently used as psychiatric or neuroprotective drugs, drugs that modulate blood flow, and mood altering drugs such as: rivastigmine, rasagiline, methylphenidate, amphetamine, tacrine, donepezil, metrifonate, fluoxetine, sertraline, paroxetine, fluvoxamine, citalopram, escitalopram, venlafaxine, nefazodone, mirtazapine, bupropion, cianopramine, femoxetine, ifoxetine, milnacipran, oxaprotiline, sibutramine, viqualine, clozapine, fenclonine, dexfenfluramine, chlorpromazine, methamphetamine, prazosin, terazosin, doxazosin, trimazosin, labetalol, medroxalol, tofenacin, trazodone, viloxazine, riluzole, and pharmacologically acceptable salts thereof, and combinations of any of the foregoing;

The HTNC agents also include molecules that are currently used as PET contrast agents, small molecules that are being used as ligands for macromolecules such as ligands for dopamine receptors and transporters, serotonin receptors and transporters, acetylcholine receptors and transporters, norepinephrine receptors and transporters, and as ligands for macromolecules that are indicators of disease such as the Beta-amyloid peptide and its imidazopyridinylbenzeneamine and benzothizolylbenzeneamine derivatives ligands, and pharmacologically acceptable salts thereof, and combinations of any of the foregoing;

The HTNC agents also include molecules that upon hydrogenation yield the above mentioned HTNCs such as (2-hydroxyethenyl)trimethylammonium chloride (that can be converted to choline by hydrogenation), (2-hydroxyethynyl)trimethylamnmollium (that can be converted to choline by two consecutive hydrogenations), (S)-2-amino-3-(5-hydroxy-1H-indol-3-yl)propenoic acid (that can be converted to 5-hydroxytryptophan by hydrogenation), (S)-2-amino-3-(3,4-dihydroxyphenyl)propenoic acid (that can be converted to L-DOPA by hydrogenation), 2-amino-2-ene-5-(diaminomethylidene amino)pentanoic acid (that can be converted to arginine by hydrogenation), 2-amino-5-(diaminomethylidene imino)pentanoic acid (that can be converted to arginine upon hydrogenation); and pharmacologically acceptable salts thereof, and combinations of any of the foregoing;

HTNC molecules are labeled with carbon-13 and nitrogen-15 at preferred positions. Preferred carbon-13 labeling positions include quaternary or tertiary non-protonated position. Labeling at non-preferred positions is sometimes added due to synthetic requirements.

Preferred Nitrogen-15 labeling positions include non-protonated positions. Labeling at non-preferred positions is sometimes added due to synthetic requirements.

Some examples of the labeled HTNCs are given in the detailed description of the invention. The numerals marking label positions are pictorially described in FIGS. 1 through 40.

One embodiment of this invention comprises detection of neurochemical metabolic pathways in the human or animal brain that were not amenable for in vivo, non-invasive investigation before, and use thereof for characterizing brain function.

A second embodiment of this invention comprises the detection of the distribution of drugs and thereby detecting the distribution of their targets (receptor, channels, and enzymes). For example Rivastigmine is known to block the cholinesterase enzyme in two places in the rat brain—the hippocampus and the cortex—in smaller quantities than in other places in the body and the brain. As a second use, in this embodiment, rivastigmine is used as a marker of specific acetylcholine esterases distribution within the brain

A third embodiment of this invention comprises simultaneous monitoring of the balance between several neurochemical agents and drugs. The neurochemicals described in this invention are given simultaneously by specific combinations to monitor the balance between the neuromodulatory systems in the individual's brain. This is a unique type of brain investigation that is enabled due to the properties of magnetic resonance spectroscopy as opposed to radioactive tracer methods (PET, SPECT). Because each neurochemical has its characteristic resonance frequencies pattern, several neurochemicals can be injected, detected and resolved simultaneously. Radioactive tracer methods are devoid of this capability because their detectors detect total radiation from a source and are usually not affected by the fine molecular structure of the source.

A forth embodiment of this invention comprises new stable-isotope-labeled isomers of known molecules. Most (but not all) of the labeled isomer-molecules that are presented here are first described and synthesized under this invention. The synthetic steps that are involved in the syntheses of these molecules are known in the art via enzymatic or organic synthetic routs or both, including synthetic routes involving hydrogenation of double and triple bonds (potentially with parahydrogen). By using synthetic precursors that are labeled with carbon-13 or nitrogen-15, the new labeled isomer-molecules are synthesized.

### DETAILED DESCRIPTION OF THE INVENTION

The following description is provided, alongside all chapters of the present invention, so as to enable any person skilled in the art to make use of the said invention and sets forth the best modes considered by the inventor for carrying out this invention. Various modifications, however, will remain apparent to those skilled in the art, since the generic principles of the present invention have been defined specifically to provide neurochemical agents, device, method and use thereof for monitoring brain activity, diagnosis of psychiatric and neurodegenerative diseases and disorders, confirmation of drug action in situ, and direct drug efficacy determination

The HTNC agents may contain non-zero nuclear spin nuclei such as carbon-13, nitrogen-15, fluorine-19, and deuterium. In this event the MR signals from which the image is generated will be substantially only from the HTNC agent itself and there will be essentially no interference from background signals (the natural abundance of ¹³C, ¹⁹F, and deuterium being negligible) and image contrast will be advantageously high. This is especially true where the HTNC agent itself is enriched above natural abundance. Thus the method according to the invention has the benefit of being able to provide significant spatial weighting to a generated image. In effect, the administration of a polarized HTNC agent to a selected region of a sample (e.g. by injection) means that the contrast effect may be localized to that region. The precise effect of course depends on the extent of distribution in the brain over the period in which the HTNC agent remains significantly polarized.

In one embodiment, a “native image” of the brain (i.e. one obtained prior to administration of the HTNC agent or one obtained for the administered HTNC agent without prior polarization as in a conventional MR experiment) may be generated to provide structural (e.g. anatomical) information upon which the image or the spectroscopic voxels obtained in the method according to the invention may be superimposed. A “native image” is generally not available where ¹³C, ¹⁵N or ¹⁹F is the imaging nucleus because of their low abundance in the body. In this case, a proton MR image may be taken to provide the anatomical information upon which the ¹³C, ¹⁵N or ¹⁹F image may be superimposed.

The HTNC agent should of course be physiologically tolerable or be capable of being provided in a physiologically tolerable, administrable form and non-toxic. Conveniently, the HTNC agent once polarized will remain so for a period sufficiently long to allow the spectroscopic/imaging procedure to be carried out in a comfortable time span. Generally sufficient polarization will be retained by the HTNC agent in its administrable form (e.g. in injection solution) if it has a T₁ value (at a field strength of 0.01-5 T and a temperature in the range 20-40° C.) of at least 2 s, preferably at least 5 s, more preferably at least 10 s, especially preferably 30 s or longer, more especially preferably 70 s or more, yet more especially preferably 100 s or more (for example at 37° C. in water at 1 T and a concentration of at least 0.1 mM). The HTNC agent may be advantageously an agent with a long T₂ relaxation time.

The long T₁ relaxation time of certain ¹³C and ¹⁵N nuclei is particularly advantageous and certain HTNC agents containing ¹³C and ¹⁵N nuclei are therefore preferred for use in the present method. The γ-factor of carbon is about ¼ of the γ-factor for hydrogen resulting in a Larmor frequency of about 10 MHz at 1 T. The RF-absorption and reflections in a patient is consequently and advantageously less than in water (proton) imaging. Preferably the polarized HTNC agent has an effective ¹³C nuclear polarization corresponding to the one obtained at thermal equilibrium at 300 K in a field of 0.1 T or more, more preferably 25 T or more, particularly preferably 100 T or more, especially preferably 5000 T or more (for example 50 kT).

When the electron cloud of a given nucleus in a certain molecule is changed due to a metabolic (chemical) process, the shielding of that atom (which is responsible for the MR signal) is changed giving rise to a shift in the MR frequency (“the chemical shift effect”). Therefore, when the molecule is metabolized, the chemical shift of a specific nucleus will change. The HTNC agents and their various metabolic products can be visualized separately using magnetic resonance spectroscopy. Either full spectrum or chemical shift selective methods may be applied. By full spectrum methods it is referred to 1D or 2D single-voxel localized spectroscopy or multi-voxel spectroscopic imaging such as methods that are based on the sequences point-resolved spectroscopy (PRESS), stimulated echo (STEAM), and single shot 2D NMR techniques. Chemical shift selective methods refer to the use of pulses sensitive to chemical shift. When the frequency difference between HTNC metabolites is 150 Hz or higher (corresponding to 3.5 ppm or higher at 1 T), the two metabolites may be excited separately and visualized in two images. Standard chemical shift selective excitation pulses may then be utilized. When the frequency separation is less, the two components may not be separated by using frequency selective RF-pulses. The phase difference created during the time delay after the excitation pulse and before the detection of the MR signal may then be used to separate the two components. It is known in the art that phase sensitive imaging pulse sequence methods may be used to generate images visualizing different metabolites. The long T₂ relaxation time which may be a characteristic of a high T₁ agent will under these circumstances make it possible to use long echo times (TE) and still get a high signal to noise ratio. Thus an important advantage of the HTNC agents used in the present method is that they exhibit a chemical shift dependent on the progress of the metabolic process.

To increase the MR signal of the HTNC agents, the present invention makes use of two methods which are known in the art as DNP and PHIP. In the DNP method, the HTNC agents are mixed with an OMRI polarization agent and frozen to 1.2° K. At this temperature the HTNC agent is of course solid. At this phase, the HTNC agents may exhibit very long T₁ relaxation times and for this reason are especially preferred for use in the present method. The T₁ relaxation time may be several hours in the bulk phase. For in vivo use, a polarized solid HTNC agent may be dissolved in administrable media (e.g. water or saline), separated from the OMRI polarization agent, and administered to a subject. In PHIP, the HTNC agents are in liquid state. After hydrogenation with parahydrogen, the HTNCs may be separated from the hydrogenation catalyst, and added to administrable media. Conventional multinuclei MR imaging is then performed according to methods that are known in the art. Thus solid HTNC agents are preferably rapidly soluble (e.g. water soluble) to assist in formulating administrable media. Preferably the HTNC agent should dissolve in a physiologically tolerable carrier (e.g. water or buffer solution) to a concentration of at least 1 mM at a rate of 1 mM/3 T₁ or more, particularly preferably 1 mM/2 T₁ or more, especially preferably 1 mM/T₁ or more. Where the solid HTNC agent is frozen, the administrable medium may be heated, preferably to an extent such that the temperature of the medium after mixing is close to 37° C.

The resulting DNP-polarized HTNC agent in liquid form may be administered either alone or with additional components such as additional HTNC agents, or agents that will prevent its degradation in the peripheral circulation, increase its blood-brain-barrier permeability, prevent its uptake by peripheral organs, or modify its effect in the brain or body.

In the PHIP method, the HTNC agent, with an unsaturated carbon-carbon bond is hydrogenated with parahydrogen in a short reaction time (less than 10 sec) with the aid of a hydrogenation catalyst. A variety of liquid state hydrogenation catalysts and asymmetric hydrogenation catalysts is known in the art. To verify the increased spin order effect, the product may be transferred to a NMR spectrometer or imager. Strong antiphase signals on proton spectra are indicative of a productive parahydrogen hydrogenation and a successful increase of the spin order. The nonequilibrium spin order obtained by hydrogenation with parahydrogen is converted to longitudinal polarization by means of a nonadiabatic field cycling. The external magnetic field is suddenly decreased and then gradually increased back to the ambient earth's magnetic field. In order to obtain a sufficiently low external magnetic field the ambient field is screened by using three concentric cylinders of magnetic field shielding known in the art as mu-metal. The field cycling is realized by dropping the sample into the magnetic shield and then gently lifting the shield. This field cycling is known in the art to result in a substantial polarization of a variety of carbon-13 labeled organic molecules.

The resulting PHIP-polarized HTNC agent in liquid form is separated from the hydrogenation catalysts. Then, the polarized agent in liquid form may be administered to the subject, either alone or with additional components such as additional HTNC agents, or agents that will prevent its degradation in the peripheral circulation, increase its blood-brain-barrier permeability, prevent its uptake by peripheral organs, or modify its effect in the brain or body.

Given that the in situ detection of the HTNC agents should be carried out within the time frame that the HTNC agent remains significantly polarized, it is desirable for administration of the polarized HTNC agent to be effected rapidly and for the MR measurement to follow shortly thereafter. The preferred administration route for the polarized HTNC agent is by bolus injection, intravenous or intra-arterial. The injection time should be equivalent to 5 T₁ or less, preferably 3 T₁ or less, particularly preferably T₁ or less, especially 0.1 T₁ or less. The HTNC agent should be preferably enriched with nuclei (e.g. ¹³C and ¹⁵N nuclei) having a long T₁ relaxation time. Preferred are ¹³C enriched high T₁ agents having ¹³C at one particular position (or more than one particular position) in an amount in excess of the natural abundance i.e. above about 1%. Preferably such a single carbon position will have 5% or more ¹³C, particularly preferably 10% or more, especially preferably 25% or more, more especially preferably 50% or more, even more preferably in excess of 99% (e.g. 99.9%). The ¹³C nuclei should preferably amount to >2% of all carbon atoms in the compound. The HTNC agent is preferably ¹³C enriched at one or more carbonyl or quaternary carbon positions, given that a ¹³C nucleus in a carbonyl group or in certain quaternary carbons may have a T₁ relaxation time typically of more than 2 s, preferably more than 5 s, especially preferably more than 30 s. Preferably the ¹³C enriched compound should be deuterium labeled, especially adjacent the ¹³C nucleus. Also preferred are HTNCs enriched with ¹³C as described above in which the ¹³C is adjacent to a ¹⁵N at a particular position. Preferably, the ¹⁵N position is enriched in an amount excess of the natural abundance i.e. above about 1%. Preferably such a single nitrogen position will have 5% or more ¹⁵N, particularly preferably 10% or more, especially preferably 25% or more, more especially preferably 50% or more, even more preferably in excess of 99% (e.g. 99.9%). Also preferred are HTNCs enriched with ¹⁵N as described above at one or more position with or without ¹³C enrichment.

It is in the scope of the present invention wherein a list of HTNCs and labeling positions are defined below in a non-limiting manner:

| | |
|---|---|
| 1) Choline | |
| a) [1-13C,15N]-choline: | HO—*CH₂—CH₂—*N(CH₃)₃ |
| b) [1-13C]-choline: | HO—*CH₂—CH₂—N(CH₃)₃ |
| c) [2-13C,15N]-choline: | HO—CH₂—*CH₂—*N(CH₃)₃ |
| d) [2-13C]-choline: | HO—CH₂—*CH₂—N(CH₃)₃ |
| e) [1,2-13C,15N]-choline: | HO—*CH₂—*CH₂—*N(CH₃)₃ |
| f) [1,2-13C]-choline: | HO—*CH₂—*CH₂—N(CH₃)₃ |
| g) [15N]-choline | HO—CH₂—CH₂—*N(CH₃)₃ |
| 2) Betaine | |
| a) [1-13C,15N]-betaine: | HO—*CO—CH₂—*N(CH₃)₃ |
| b) [1-13C]-betaine: | HO—*CO—CH₂—N(CH₃)₃ |
| c) [2-13C,15N]-betaine: | HO—CO—*CH₂—*N(CH₃)₃ |
| d) [2-13C]-betaine: | HO—CO—*CH₂—N(CH₃)₃ |
| e) [1,2-13C,15N]-betaine: | HO—*CO—*CH₂—*N(CH₃)₃ |
| f) [1,2-13C]-betaine: | HO—*CO—*CH₂—N(CH₃)₃ |
| g) [15N]-betaine: | HO—CO—CH₂—*N(CH₃)₃ |
| 3) Acetylcholine | |
| a) [1-13C,15N]-acetylcholine: | CH₃COO*CH₂CH₂*N(CH₃)₃ |
| b) [1-13C]-acetylcholine: | CH₃COO*CH₂CH₂N(CH₃)₃ |
| c) [2-13C,15N]-acetylcholine: | CH₃COOCH₂*CH₂*N(CH₃)₃ |
| d) [2-13C]-acetylcholine: | CH₃COOCH₂*CH₂N(CH₃)₃ |
| e) [1,2-13C,15N]-acetylcholine: | CH₃COO*CH₂*CH₂*N(CH₃)₃ |
| f) [1,2-13C]-acetylcholine: | CH₃COO*CH₂*CH₂N(CH₃)₃ |
| g) [3-13C,15N]-acetylcholine: | CH₃*COOCH₂CH₂*N(CH₃)₃ |
| h) [3-13C]-acetylcholine: | CH₃*COOCH₂CH₂N(CH₃)₃ |
| i) [1,3-13C,15N]-acetylcholine: | CH₃*COO*CH₂CH*N(CH₃)₃ |
| j) [1,3-13C]-acetylcholine: | CH₃*COO*CH₂CH₂N(CH₃)₃ |
| k) [2,3-13C,15N]-acetylcholine: | CH₃*COOCH₂*CH₂*N(CH₃)₃ |
| l) [2,3-13C]-acetylcholine: | CH₃*COOCH₂*CH₂N(CH₃)₃ |
| m) [1,2,3-13C,15N]-acetylcholine: | CH₃*COO*CH₂*CH₂*N(CH₃)₃ |
| n) [1,2,3-13C]-acetylcholine: | CH₃*COO*CH₂*CH₂N(CH₃)₃ |
| o) [15N]-acetylcholine: | CH₃COOCH₂CH₂*N(CH₃)₃ |
| 4) Acetate | |
| a) [1-13C]-acetate: | HO*COCH3 |
| 5) Aspartate | |
| a) [1-13C]-aspartate: | HOOC*CH(NH₂)CH₂COOH |
| b) [2-13C]-aspartate: | HOOCCH(NH₂)*CH₂COOH |
| c) [3-13C]-aspartate: | HOOCCH(NH₂)CH₂*COOH |
| d) [4-13C]-aspartate: | HOO*CCH(NH₂)CH₂COOH |
| e) [1,2-13C]-aspartate: | HOOC*CH(NH₂)*CH₂COOH |
| f) [2,3-13C]-aspartate: | HOOCCH(NH₂)*CH₂*COOH |
| g) [2,4-13C]-aspartate: | HOO*CCH(NH₂)*CH₂COOH |
| h) [1,3-13C]-aspartate: | HOOC*CH(NH₂)CH₂*COOH |
| i) [1,4-13C]-aspartate: | HOO*C*CH(NH₂)CH₂COOH |
| j) [3,4-13C]-aspartate: | HOO*CCH(NH₂)CH₂*COOH |
| k) [1,3,4-13C]-aspartate: | HOO*C*CH(NH₂)CH₂*COOH |
| l) [1,2,3-13C]-aspartate: | HOOC*CH(NH₂)*CH₂*COOH |
| m) [2,3,4-13C]-aspartate: | HOO*CCH(NH₂)*CH₂*COOH |
| n) [1,2,4-13C]-aspartate: | HOO*C*CH(NH₂)*CH₂COOH |
| o) [1,2,3,4-13C]-aspartate: | HOO*C*CH(NH₂)*CH₂*COOH |
| 6) N-acetylaspartate | |
| a) [4-13C]-N-acetylaspartate: | HOO*CCH(NH(COCH3))CH₂COOH |
| b) [5-13C]-N-acetylaspartate: | HOOCCH(NH(*COCH3))CH₂COOH |
| c) [3-13C]-N-acetylaspartate: | HOOCCH(NH(COCH3))CH₂*COOH |
| d) [3,4-13C]-N-acetylaspartate: | HOO*CCH(NH(COCH3))CH₂*COOH |
| e) [3,5-13C]-N-acetylaspartate: | HOOCCH(NH(*COCH3))CH₂*COOH |
| f) [4,5-13C]-N-acetylaspartate: | HOO*CCH(NH(*COCH3))CH₂COOH |
| g) [3,4,5-13C]-N-acetylaspartate: | HOO*CCH(NH(*COCH3))CH₂*COOH |
| h) ¹⁵N-acetylaspartate: | HOOCCH(*NH(COCH3))CH₂COOH |
| i) [5-13C,15N]-N-acetylaspartate: | HOOCCH(*NH(*COCH3))CH₂COOH |
| 7) Creatine | |
| a) [13C₄,15N₃]-creatine: | H₂*N^{+*}C(*NH₂)*N(*CH₃)*CH₂*CO₂⁻ |
| b) [4-13C]-creatine: | H₂N^{+*}C(NH₂)N(CH₃)CH₂CO₂⁻ |
| c) [1-13C]-creatine: | H₂N⁺C(NH₂)N(CH₃)CH₂*CO₂⁻ |
| d) [1,4-13C]-creatine: | H₂N^{+*}C(NH₂)N(CH₃)CH₂*CO₂⁻ |
| e) [4-13C,3-15N]-creatine: | H₂N^{+*}C(NH₂)*N(CH₃)CH₂CO₂⁻ |
| f) [1-13C,3-15N]-creatine: | H₂N⁺C(NH₂)*N(CH₃)CH₂*CO₂⁻ |
| g) [1,4-13C,3-15N]-creatine: | H₂N^{+*}C(NH₂)*N(CH₃)CH₂*CO₂⁻ |
| h) [3-15N]-creatine: | H₂N⁺C(NH₂)*N(CH₃)CH₂CO₂⁻ |
| 8) L-Tyrosine | |
| a) [9-13C]-L-tyrosine: | 4-HO—C₆H₄CH₂CH(NH₂)*COOH |
| b) [8,9-13C]-L-tyrosine: | 4-HO—C₆H₄CH₂*CH(NH₂)*COOH |
| c) [1,8,9-13C]-L-tyrosine: | 4-HO—*C₆H₄CH₂*CH(NH₂)*COOH(phenyl-1-¹³C) |
| d) [1,4,8,9-13C]-L-tyrosine: | 4-HO—*C₆H₄CH₂*CH(NH₂)*COOH(phenyl-1,4-¹³C₂) |
| e) [1,3,4,8,9-13C]-L-tyrosine: | 4-HO—*C₆H₄CH₂*CH(NH₂)*COOH(phenyl-1,3,4-¹³C₃) |
| f) [1,2,3,4,5,6,8,9-13C]-L-tyrosine: | 4-HO—*C₆H₄CH₂*CH(NH₂)*COOH(phenyl-¹³C₆) |
| g) [1-13C]-L-tyrosine: | 4-HO—*C₆H₄CH₂CH(NH₂)COOH(phenyl-¹³C₁) |
| h) [4-13C]-L-tyrosine: | 4-HO—*C₆H₄CH₂CH(NH₂)COOH(phenyl-¹³C₁) |
| i) [13C₉]-L-tyrosine: | 4-HO—*C₆H₄CH₂*CH(NH₂)*COOH(phenyl-¹³C₆) |
| 9) 3-(3,4-Dihydroxyphenyl)-alanine (L-DOPA) | |
| a) [9-13C]-L-DOPA: | 3-HO—,4HO—C₆H₃CH₂CH(NH₂)*COOH |
| b) [8,9-13C]-L-DOPA: | 3-HO—,4HO—C₆H₃CH₂*CH(NH₂)*COOH |
| c) [1,8,9-13C]-L-DOPA: | 3-HO—,4HO—*C₆H₃CH₂*CH(NH₂)*COOH(phenyl-1-¹³C₁) |
| d) [1,4,8,9-13C]-L-DOPA: | 3-HO—,4HO—*C₆H₃CH₂*CH(NH₂)*COOH(phenyl-1,4-¹³C₂) |
| e) [1,3,4,8,9-13C]-L-DOPA: | 3-HO—,4HO—*C₆H₃CH₂*CH(NH₂)*COOH(phenyl-1,3,4-¹³C₃) |
| f) [1,3,4-13C]-L-DOPA: | 3-HO—,4HO—*C₆H₃CH₂CH(NH₂)COOH(phenyl-1,3,4-¹³C₃) |
| g) [1,2,3,4,5,6,8,9-13C]-L-DOPA: | 3-HO—,4HO—*C₆H₃CH₂*CH(NH₂)*COOH(phenyl-¹³C₆) |
| h) [1,2,3,4,5,6-13C]-L-DOPA: | 3-HO—,4HO—*C₆H₃CH₂CH(NH₂)COOH(phenyl-¹³C₆) |
| i) [3-13C]-L-DOPA: | 3-HO—,4HO—*C₆H₃CH₂CH(NH₂)COOH(phenyl-¹³C₁) |
| j) [4-13C]-L-DOPA: | 3-HO—,4HO—*C₆H₃CH₂CH(NH₂)COOH(phenyl-¹³C₁) |
| k) [1-13C]-L-DOPA: | 3-HO—,4HO—*C₆H₃CH₂CH(NH₂)COOH(phenyl-¹³C₁) |
| l) [13C₉]-L-DOPA: | 3-HO—,4HO—*C₆H₃CH₂*CH(NH₂)*COOH(phenyl-¹³C₆) |
| m) [8-13C]-L-DOPA: | 3-HO—,4HO—C₆H₃CH₂*CH(NH₂)COOH |
| 10) Dopamine | |
| a) [13C₆]-dopamine: | 3-HO—,4HO—*C₆H₃CH₂CH₂—NH₂(phenyl-¹³C₆) |
| b) [1-13C]-dopamine: | 3-HO—,4HO—*C₆H₃CH₂CH₂—NH₂(phenyl-¹³C₁) |
| c) [3-13C]-dopamine: | 3-HO—,4HO—*C₆H₃CH₂CH₂—NH₂(phenyl-¹³C₁) |
| d) [4-13C]-dopamine: | 3-HO—,4HO—*C₆H₃CH₂CH₂—NH₂(phenyl-¹³C₁) |
| e) [1,4-13C]-dopamine: | 3-HO—,4HO—*C₆H₃CH₂CH₂—NH₂(phenyl-¹³C₂) |
| f) [1,3-13C]-dopamine: | 3-HO—,4HO—*C₆H₃CH₂CH₂—NH₂(phenyl-¹³C₂) |
| g) [3,4-13C]-dopamine: | 3-HO—,4HO—*C₆H₃CH₂CH₂—NH₂(phenyl-¹³C₂) |
| h) [1,3,4-13C]-dopamine: | 3-HO—,4HO—*C₆H₃CH₂CH₂—NH₂(phenyl-¹³C₃) |
| 11) Norepinephrine | |
| a) [13C₆]-norepinephrine: | 3-HO—,4HO—*C₆H₃CH(OH)CH₂—NH₂(phenyl-¹³C₆) |
| b) [1-13C]-norepinephrine: | 3-HO—,4HO—*C₆H₃CH(OH)CH₂—NH₂(phenyl-¹³C₁) |
| c) [3-13C]-norepinephrine: | 3-HO—,4HO—*C₆H₃CH(OH)CH₂—NH₂(phenyl-¹³C₁) |
| d) [4-13C]-norepinephrine: | 3-HO—,4HO—*C₆H₃CH(OH)CH₂—NH₂(phenyl-¹³C₁) |
| 12) Epinephrine | |
| a) [13C₆]-epinephrine: | 3-HO—,4HO—*C₆H₃CH(OH)CH₂—NH(CH)₃(phenyl-¹³C₆) |
| b) [1-13C]-epinephrine: | 3-HO—,4HO—*C₆H₃CH(OH)CH₂—NH(CH)₃(phenyl-¹³C₁) |
| c) [3-13C]-epinephrine: | 3-HO—,4HO—*C₆H₃CH(OH)CH₂—NH(CH)₃(phenyl-¹³C₁) |
| d) [4-13C]epinephrine: | 3-HO—,4HO—*C₆H₃CH(OH)CH₂—NH(CH)₃(phenyl-¹³C₁) |
| 13) Vanillylmandelic acid (VMA) | |
| a) [13C₆]-VMA: | 3-HO—,4HO—*C₆CH(OH)CO₂H(phenyl-¹³C₆) |
| b) [8-13C]-VMA: | 3-HO—,4HO—C₆H₃CH(OH)*CO₂H |
| c) [13C₈]-VMA: | 3-HO—,4HO—*C₆H₃*CH(OH)*CO₂H |
| d) [13C₇]-VMA: | 3-HO—,4HO—*C₆H₃CH(OH)*CO₂H(phenyl-¹³C₆) |
| 14) Homovanillic acid (HVA) | |
| a) [13C₆]-HVA: | 3-HO—,4HO—*C₆H₃CH₂CO₂H(phenyl-¹³C₆) |
| b) [13C₈]-HVA: | 3-HO—,4HO—*C₆H₃*CH₂*CO₂H(phenyl-¹³C₆) |
| c) [13C₇]-HVA: | 3-HO—,4HO—*C₆H₃CH₂*CO₂H(phenyl-¹³C₆) |
| d) [8-13C]-HVA: | 3-HO—,4HO—C₆H₃CH₂*CO₂H |
| 15) 3-O-methyldopamine (3OMD) | |
| a) [13C₆]-3OMD: | 3-CH₃O—,4HO—*C₆H₃CH₂CH₂NH₂(phenyl-¹³C₆) |
| b) [13C₈]-3OMD: | 3-CH₃O—,4HO—*C₆H₃*CH₂*CH₂NH₂(phenyl-¹³C₆) |
| c) [1,3-13C]-3OMD: | 3-CH₃O—,4HO—*C₆H₃CH₂CH₂NH₂(phenyl-¹³C₂) |
| d) [1,3,4-13C]-3OMD: | 3-CH₃O—,4HO—*C₆H₃CH₂CH₂NH₂(phenyl-¹³C₃) |
| 16) 3-O-methylnorepinephrine (3OMN) | |
| a) [13C₆]-3OMN: | 3-CH₃O—,4HO—*C₆H₃CH(OH)CH₂NH₂(phenyl-¹³C₆) |
| b) [13C₈]-3OMN: | 3-CH₃O—,4HO—*C₆H₃*CH(OH)*CH₂NH₂(phenyl-¹³C₆) |
| c) [1,3-13C]-3OMN: | 3-CH₃O—,4HO—*C₆H₃CH(OH)CH₂NH₂(phenyl-¹³C₂) |
| d) [1,3,4-13C]-3OMN: | 3-CH₃O—,4HO—*C₆H₃CH(OH)CH₂NH₂(phenyl-¹³C₃) |
| 17) 3-O-methylepinephrine (3OME) | |
| a) [13C₆]-3OME: | 3-CH₃O—,4HO—*C₆H₃CH(OH)CH₂NH(CH₃)(phenyl-¹³C₆) |
| b) [13C₈]-3OME: | 3-CH₃O—,4HO—*C₆H₃*CH(OH)*CH₂NH(CH₃)(phenyl-¹³C₆) |
| c) [1,3-13C]-3OME: | 3-CH₃O—,4HO—*C₆H₃CH(OH)CH₂NH(CH₃)(phenyl-¹³C₂) |
| d) [1,3,4-13C]-3OME: | 3-CH₃O—,4HO—*C₆H₃CH(OH)CH₂NH(CH₃)(phenyl-¹³C₃) |
| 18) Dopaquinone | |
| a) [13C₉]-dopaquinone: | 3O—,4O—*C₆H₃*CH₂*CH(NH₂)*COOH(phenyl-¹³C₆) |
| b) [1,3,4,8,9-13C]-dopaquinone: | 3O—,4O—*C₆H₃CH₂*CH(NH₂)*COOH(phenyl-¹³C₃) |
| c) [1-13C]-dopaquinone: | 3O—,4O—*C₆H₃CH₂CH(NH₂)COOH(phenyl-¹³C₁) |
| d) [3-13C]-dopaquinone: | 3O—,4O—*C₆H₃CH₂CH(NH₂)COOH(phenyl-¹³C₁) |
| e) [4-13C]-dopaquinone: | 3O—,4O—*C₆H₃CH₂CH(NH₂)COOH(phenyl-¹³C₁) |
| f) [8-13C]-dopaquinone: | 3O—,4O—C₆H₃CH₂*CH(NH₂)COOH |
| g) [9-13C]-dopaquinone: | 3O—,4O—C₆H₃CH₂CH(NH₂)*COOH |
| h) [13C₆]-dopaquinone: | 3O—,4O—*C₆H₃CH₂CH(NH₂)COOH(phenyl-¹³C₆) |
| 19) L-Tryptophan | |
| a) [13C₁₁]-L-tryptophan: | *C₆H₄*C(*CH₂*CH(NH₂)*COOH)*CH—NH(phenyl-¹³C₆) |
| b) [13C₁₁, 15N]-L-tryptophan: | *C₆H₄*C(*CH₂*CH(NH₂)*COOH)*CH—*NH(phenyl-¹³ C₆) |
| c) [13C₆]-L-tryptophan: | *C₆H₄C(CH₂CH(NH₂)COOH)CH—NH(phenyl-¹³C₆) |
| d) [1,2,3,8,10,11-13C₁₁]-L-tryptophan: | *C₆H₄*C(CH₂*CH(NH₂)*COOH)*CH—NH |
| e) [1-13C₁₁]-L-tryptophan: | C₆H₄C(CH₂CH(NH₂)COOH)*CH—NH |
| f) [2-13C₁₁]-L-tryptophan: | C₆H₄*C(CH₂CH(NH₂)COOH)CH—NH |
| g) [3-13C₁₁]-L-tryptophan: | *C₆H₄C(CH₂CH(NH₂)COOH)CH—NH |
| h) [8-13C₁₁]-L-tryptophan: | *C₆H₄C(CH₂CH(NH₂)COOH)CH—NH |
| i) [10-13C₁₁]-L-tryptophan: | C₆H₄C(CH₂*CH(NH₂)COOH)CH—NH |
| j) [11-13C₁₁]-L-tryptophan: | C₆H₄C(CH₂CH(NH₂)*COOH)CH—NH |
| k) [1,2,3,8,10,11-13C₁₁, 15N]-L-tryptophan: | *C₆H₄*C(CH₂*CH(NH₂)*COOH)*CH—*NH |
| l) [1-13C₁₁, 15N]-L-tryptophan: | C₆H₄C(CH₂CH(NH₂)COOH)*CH—*NH |
| m) [2-13C₁₁, 15N]-L-tryptophan: | C₆H₄*C(CH₂CH(NH₂)COOH)CH—*NH |
| n) [3-13C₁₁, 15N]-L-tryptophan: | *C₆H₄C(CH₂CH(NH₂)COOH)CH—*NH |
| o) [8-13C₁₁, 15N]-L-tryptophan: | *C₆H₄C(CH₂CH(NH₂)COOH)CH—*NH |
| p) [10-13C₁₁, 15N]-L-tryptophan: | C₆H₄C(CH₂*CH(NH₂)COOH)CH—*NH |
| q) [11-13C₁₁, 15N]-L-tryptophan: | C₆H₄C(CH₂CH(NH₂)*COOH)CH—*NH |
| 20) 5-hydroxy-tryptophan | |
| a) [13C₁₁]-5-hydroxy-tryptophan: | 5-OH—*C₆H₃*C(*CH₂*CH(NH₂)*COOH)*CH—NH(phenyl-¹³C₆) |
| b) [13C₁₁,15N]-5-hydroxy-tryptophan: | 5-OH*C₆H₃*C(*CH₂*CH(NH₂)*COOH)*CH*NH(phenyl-¹³C₆) |
| c) [13C₆]-5-hydroxy-tryptophan: | 5-OH*C₆H₃C(CH₂CH(NH₂)COOH)CHNH(phenyl-¹³C₆) |
| d) [1,2,3,5,8,10,11-13C]-5-hydroxy-tryptophan: | 5-OH*C₆H₃*C(CH₂*CH(NH₂)*COOH)*CH—NH |
| e) [1-13C]-5-hydroxy-tryptophan: | 5-OH—C₆H₃C(CH₂CH(NH₂)COOH)*CH—NH |
| f) [2-13C]-5-hydroxy-tryptophan: | 5-OH—C₆H₃*C(CH₂CH(NH₂)COOH)CH—NH |
| g) [3-13C]-5-hydroxy-tryptophan: | 5-OH—*C₆H₃C(CH₂CH(NH₂)COOH)CH—NH |
| h) [5-13C]-5-hydroxy-tryptophan: | 5-OH—*C₆H₃C(CH₂CH(NH₂)COOH)CH—NH |
| i) [8-13C]5-hydroxy-tryptophan: | 5-OH—*C₆H₃C(CH₂CH(NH₂)COOH)CH—NH |
| j) [10-13C]5-hydroxy-tryptophan: | 5-OH—C₆H₃C(CH₂*CH(NH₂)COOH)CH—NH |
| k) [11-13C]-5-hydroxy-tryptophan: | 5-OH—C₆H₃C(CH₂CH(NH₂)*COOH)CH—NH |
| l) [1,2,3,5,8,10,11-13C,15N]-5-hydroxy-tryptophan: | 5-OH—*C₆H₃*C(CH₂*CH(NH₂)*COOH)*CH—*NH |
| m) [1-13C,15N]-5-hydroxy-tryptophan: | 5-OH—C₆H₃C(CH₂CH(NH₂)COOH)*CH—*NH |
| n) [2-13C,15N]-5-hydroxy-tryptophan: | 5-OH—C₆H₃*C(CH₂CH(NH₂)COOH)CH—*NH |
| o) [3-13C,15N]-5-hydroxy-tryptophan: | 5-OH—*C₆H₃C(CH₂CH(NH₂)COOH)CH—*NH |
| p) [5-13C,15N]-5-hydroxy-tryptophan: | 5-OH—*C₆H₃C(CH₂CH(NH₂)COOH)CH—*NH |
| q) [8-13C,15N]-5-hydroxy-tryptophan: | 5-OH—*C₆H₃C(CH₂CH(NH₂)COOH)CH—*NH |
| r) [10-13C,15N]-5-hydroxy-tryptophan: | 5-OH—C₆H₃C(CH₂*CH(NH₂)COOH)CH—*NH |
| s) [11-13C,15N]-5-hydroxy-tryptophan: | 5-OH—C₆H₃C(CH₂CH(NH₂)*COOH)CH*NH |
| t) [1,2,3,4,5,6,7,8-13C,15N]-5-hydroxy-tryptophan: | 5-OH*C₆H₃*C(CH₂CH(NH₂)COOH)*CH*NH(phenyl-¹³C₁) |
| 21) 5-hydroxy-tryptamine (5-HT), serotonin | |
| a) [13C₁₀]-serotonin: | 5-OH—*C₆H₃*C(*CH₂*CH₂NH₂)*CH—NH(phenyl-¹³C₆) |
| b) [13C₁₀,15N]-serotonin: | 5-OH—*C₆H₃*C(*CH₂*CH₂NH₂)*CH—*NH(phenyl-¹³C₆) |
| c) [13C₆]-serotonin: | 5-OH—*C₆H₃C(CH₂CH₂NH₂)CH—NH(phenyl-¹³C₆) |
| d) [1,2,3,5,8-13C]-serotonin: | 5-OH—*C₆H₃*C(CH₂CH₂NH₂)*CH—NH(phenyl-¹³C₃) |
| e) [1-13C]-serotonin: | 5-OH—C₆H₃—C(CH₂CH₂NH₂)*CH—NH |
| f) [2-13C]-serotonin: | 5-OH—C₆H₃—*C(CH₂CH₂NH₂)CH—NH |
| g) [3-13C]-serotonin: | 5-OH—*C₆H₃—C(CH₂CH₂NH₂)CH—NH(phenyl-¹³C₁) |
| h) [5-13C]-serotonin: | 5-OH—*C₆H₃—C(CH₂CH₂NH₂)CH—NH(phenyl-¹³C₁) |
| i) [8-13C]-serotonin: | 5-OH—*C₆H₃—C(CH₂CH₂NH₂)CH—NH(phenyl-¹³C₁) |
| j) [1,2,3,5,8-13C,15N]-serotonin: | 5-OH—*C₆H₃*C(CH₂CH₂NH₂)*CH—*NH(phenyl-¹³C₃) |
| k) [1-13C,15N]-serotonin: | 5-OH—C₆H₃—C(CH₂CH₂NH₂)*CH—*NH |
| l) [2-13C,15N]-serotonin: | 5-OH—C₆H₃—*C(CH₂CH₂NH₂)CH—*NH |
| m) [3-13C,15N]-serotonin: | 5-OH—*C₆H₃—C(CH₂CH₂NH₂)CH—*NH(phenyl-¹³C₁) |
| n) [5-13C,15N]-serotonin: | 5-OH—*C₆H₃—C(CH₂CH₂NH₂)CH—*NH(phenyl-¹³C₁) |
| o) [8-13C,15N]-serotonin: | 5-OH—*C₆H₃—C(CH₂CH₂NH₂)CH—*NH(phenyl-¹³C₁) |
| p) [2,8-13C,15N]-serotonin: | 5-OH—*C₆H₃—*C(CH₂CH₂NH₂)CH—*NH(phenyl-¹³C₁) |
| 22) 5-hydroxyindole acetaldehyde (5-HIA) | |
| a) [13C₁₀]-5-HIA: | 5-OH—*C₆H₃*C(*CH₂*CHO)*CH—NH(phenyl-¹³C₆) |
| b) [13C₁₀,15N]-5-HIA: | 5-OH—*C₆H₃*C(*CH₂*CHO)*CH—*NH(phenyl-¹³C₆) |
| c) [13C₆]-5-HIA: | 5-OH—*C₆H₃C(CH₂CHO)CH—NH(phenyl-¹³C₆) |
| d) [1,2,3,5,8,10-13C₁₀]-5-HIA: | 5-OH—*C₆H₃*C(CH₂*CHO)*CH—NH(phenyl-¹³C₃) |
| e) [1-13C₁₀]-5-HIA: | 5-OH—C₆H₃C(CH₂CHO)*CH—NH |
| f) [2-13C₁₀]-5-HIA: | 5-OH—C₆H₃*C(CH₂CHO)CH—NH |
| g) [3-13C₁₀]-5-HIA: | 5-OH—*C₆H₃C(CH₂CHO)CH—NH(phenyl-¹³C₁) |
| h) [5-13C₁₀]-5-HIA: | 5-OH—*C₆H₃C(CH₂CHO)CH—NH(phenyl-¹³C₁) |
| i) [8-13C₁₀]-5-HIA: | 5-OH—*C₆H₃C(CH₂CHO)CH—NH(phenyl-¹³C₁) |
| j) [10-13C₁₀]-5-HIA: | 5-OH—C₆H₃C(CH₂*CHO)CH—NH |
| k) [1,2,3,5,8,10-13C₁₀,15N]-5-HIA: | 5-OH—*C₆H₃*C(CH₂*CHO)*CH—*NH(phenyl-¹³C₃) |
| l) [1-13C₁₀,15N]-5-HIA: | 5-OH—C₆H₃C(CH₂CHO)*CH—*NH |
| m) [2-13C₁₀,15N]-5-HIA: | 5-OH—C₆H₃*C(CH₂CHO)CH—*NH |
| n) [3-13C₁₀,15N]-5-HIA: | 5-OH—*C₆H₃C(CH₂CHO)CH—*NH(phenyl-¹³C₁) |
| o) [5-13C₁₀,15N]-5-HIA: | 5-OH—*C₆H₃C(CH₂CHO)CH—*NH(phenyl-¹³C₁) |
| p) [8-13C₁₀,15N]-5-HIA: | 5-OH—*C₆H₃C(CH₂CHO)CH—*NH(phenyl-¹³C₁) |
| q) [10-13C₁₀,15N]-5-HIA: | 5-OH—C₆H₃C(CH₂*CHO)CH—*NH |
| 23) 5-Hydroxyindole acetic acid (5-HIAA) | |
| a) [13C₁₀]-5-HIAA: | 5-OH—*C₆H₃*C(*CH₂*CO₂H)*CH—NH(phenyl-¹³C₆) |
| b) [13C₁₀,15N]-5-HIAA: | 5-OH—*C₆H₃*C(*CH₂*CO₂H)*CH—*NH(phenyl-¹³C₆) |
| c) [13C₁₀]-5-HIAA: | 5-OH—*C₆H₃*C(*CH₂*CO₂H)*CH—NH(phenyl-¹³C₆₎ |
| d) [1,2,3,5,8,10-13C₁₀]-5-HIAA: | 5-OH—*C₆H₃*C(CH₂*CO₂H)*CH—NH(phenyl-¹³C₃) |
| e) [1-13C₁₀]-5-HIAA: | 5-OH—C₆H₃C(CH₂CO₂H)*CH—NH |
| f) [2-13C₁₀]-5-HIAA: | 5-OH—C₆H₃*C(CH₂CO₂H)CH—NH |
| g) [3-13C₁₀]-5-HIAA: | 5-OH—*C₆H₃C(CH₂CO₂H)CH—NH(phenyl-¹³C₁) |
| h) [5-13C₁₀]-5-HIAA: | 5-OH—*C₆H₃C(CH₂CO₂H)CH—NH(phenyl-¹³C₁) |
| i) [8-13C₁₀]-5-HIAA: | 5-OH—*C₆H₃C(CH₂CO₂H)CH—NH(phenyl-¹³C₁) |
| j) [10-13C₁₀]-5-HIAA: | 5-OH—C₆H₃C(CH₂*CO₂H)CH—NH |
| k) [1,2,3,5,8,10-13C₁₀,15N]-5-HIAA: | 5-OH—*C₆H₃*C(CH₂*CO₂H)*CH—*NH(phenyl-¹³C₃) |
| l) [1-13C₁₀,15N]-5-HIAA: | 5-OH—C₆H₃C(CH₂CO₂H)*CH—*NH |
| m) [2-13C₁₀,15N]-5-HIAA: | 5-OH—C₆H₃*C(CH₂CO₂H)CH—*NH |
| n) [3-13C₁₀,15N]-5-HIAA: | 5-OH—*C₆H₃C(CH₂CO₂H)CH—*NH(phenyl-¹³C₁) |
| o) [5-13C₁₀,15N]-5-HIAA: | 5-OH—*C₆H₃C(CH₂CO₂H)CH—*NH(phenyl-¹³C₁) |
| p) [8-13C₁₀,15N]-5-HIAA: | 5-OH—*C₆H₃C(CH₂CO₂H)CH—*NH(phenyl-¹³C₁) |
| q) [10-13C₁₀,15N]-5-HIAA: | 5-OH—C₆H₃C(CH₂*CO₂H)CH—*NH |
| 24) Melatonin | |
| a) [13C₁₂]-melatonin: | 5-*CH₃O—*C₆H₃*C(*CH₂*CH₂NH*CO*CH₃)*CH—NH(phenyl-¹³C₃) |
| b) [13C₆]-melatonin: | 5-CH₃O—*C₆H₃C(CH₂CH₂NHCOCH₃)CH—NH(phenyl-¹³C₆) |
| c) [2-13C]-melatonin: | 5-CH₃O—C₆H₃*C(CH₂CH₂NHCOCH₃)CH—NH |
| d) [1-13C]-melatonin: | 5-CH₃O—C₆H₃C(CH₂CH₂NHCOCH₃)*CH—NH |
| e) [11-13C]-melatonin: | 5-CH₃O—C₆H₃C(CH₂CH₂NH*COCH₃)CH—NH |
| f) [13C₁₂,15N]-melatonin: | 5-*CH₃O—*C₆H₃*C(*CH₂*CH₂NH*CO*CH₃)*CH—*NH(phenyl-¹³C₃) |
| g) [13C₆,15N]-melatonin: | 5-CH₃O—*C₆H₃C(CH₂CH₂NHCOCH₃)CH—*NH(phenyl-¹³C₆) |
| h) [2-13C,15N]-melatonin: | 5-CH₃O—C₆H₃*C(CH₂CH₂NHCOCH₃)CH—*NH |
| i) [1-13C,15N]-melatonin: | 5-CH₃O—C₆H₃C(CH₂CH₂NHCOCH₃)*CH—*NH |
| j) [11-13C,15N]-melatonin: | 5-CH₃O—C₆H₃C(CH₂CH₂NH*COCH₃)CH—*NH |
| 25) Glutamate | |
| a) [1-13C]-glutamate: | HOO*CCH₂CH₂CHC(NH₂)OOH |
| b) [5-13C]-glutamate: | HOOCCH₂CH₂CH*C(NH₂)OOH |
| c) [1,5-13C]-glutamate: | HOO*CCH₂CH₂CH*C(NH₂)OOH |
| 26) Gamma-aminobutyric acid | |
| a) [1-13C]-gamma-aminobutyric acid: | H₂N(CH₂)₃*COOH |
| b) [13C₄]-gamma-aminobutyric acid: | H₂N(*CH₂)₃*COOH |
| 27) Rivastigmine tartrate | |
| a) [15N₂]-rivastigmine tartrate | |
| b) [5-13C]-rivastigmine tartrate | |
| c) [5-13C,3-15N]-rivastigmine tartrate | |
| d) [5-13C,15N₂]-rivastigmine tartrate | |
| e) [13C₆(phenyl)]-rivastigmine tartrate | |
| f) [13C₁₄]-rivastigmine tartrate | |
| g) [13C₁₄,15N₂]-rivastigmine tartrate | |
| 28) Rasagiline (N-propargyl-1-(R)aminoindan) | |
| a) [1,2-13C]-rasagiline | |
| b) [2-13C]-rasagiline | |
| c) [13C₁₂]-rasagiline | |
| d) [phenyl-13C₆]-rasagiline: | [7,8,9,10,11,12-13C]-rasagiline |
| 29) Methylphenidate (methyl 2-phenyl-2-(2-piperidyl)acetate) | |
| a) [1-13C]-methylphenidate | |
| b) [1,2-13C]-methylphenidate | |
| c) [2-13C]-methylphenidate | |
| d) [3,4,5,6,7,8-13C]-methylphenidate | |
| e) [1,2,3,4,5,6,7,8-13C]-methylphenidate | |
| f) [1,2,3,4,5,6,7,8,14-13C]-methylphenidate | |
| g) [13C₁₄]-methylphenidate | |
| 30) Amphetamine (alpha-methyl-phenethylamine) | |
| a) [phenyl-13C₆]-amphetamine | |
| 31) Imidazopyridinylbenzeneamine derivatives | |
| a) [9-13C]-imidazopyridinylbenzeneamine | |
| b) [11-13C]-imidazopyridinylbenzeneamine | |
| c) [2-15N]-imidazopyridinylbenzeneamine | |
| d) [8-15N]-imidazopyridinylbenzeneamine | |
| e) [7-13C,2,8-15N]-imidazopyridinylbenzeneamine | |
| 32) Benzothizolylbenzeneamine derivatives | |
| a) [9-13C]-benzothizolylbenzeneamine | |
| b) [11-13C]-benzothizolylbenzeneamine | |
| c) [7-13C,8-15N]-benzothizolylbenzeneamine | |
| 33) (2-hydroxyethenyl)trimethylammonium | |
| a) [1-13C,15N]-(2-hydroxyethenyl)trimethylammonium: | HO*CHCH*N(CH₃)₃ |
| b) [2-13C,15N]-(2-hydroxyethenyl)trimethylammonium: | HOCH*CH*N(CH₃)₃ |
| c) [1,2-13C,15N]-(2-hydroxyethenyl)trimethylammonium: | HO*CH*CH*N(CH₃)₃ |
| d) [1-13C]-(2-hydroxyethenyl)trimethylammonium: | HO*CHCHN(CH₃)₃ |
| e) [2-13C]-(2-hydroxyethenyl)trimethylammonium: | HOCH*CHN(CH₃)₃ |
| f) [1,2-13C]-(2-hydroxyethenyl)trimethylammonium: | HO*CH*CHN(CH₃)₃ |
| 34) (2-hydroxyethynyl)trimethylammonium | |
| a) [1-13C,15N]-(2-hydroxyethynyl)trimethylammonium: | HO*CC*N(CH₃)₃ |
| b) [2-13C,15N]-(2-hydroxyethynyl)trimethylammonium: | HOC*C*N(CH₃)₃ |
| c) [1,2-13C,15N]-(2-hydroxyethynyl)trimethylammonium: | HO*C*C*N(CH₃)₃ |
| d) [1-13C]-(2-hydroxyethynyl)trimethylammonium: | HO*CCN(CH₃)₃ |
| e) [2-13C]-(2-hydroxyethynyl)trimethylammonium: | HOC*CN(CH₃)₃ |
| f) [1,2-13C]-(2-hydroxyethynyl)trimethylammonium: | HO*C*CN(CH₃)₃ |
| 35) (S)-2-amino-3-(5-hydroxy-1H-indol-3-yl)propenoic acid | |
| a) [9-13C]-(S)-2-amino-3-(5-hydroxy-1H-indol-3-yl)propenoic acid: | 5-OHC₆H₃C(*CHC(NH₂)COOH)CHNH |
| b) [10-13C]-(S)-2-amino-3-(5-hydroxy-1H-indol-3-yl)propenoic acid: | 5-OHC₆H₃C(CH*C(NH₂)COOH)CHNH |
| c) [8-13C]-(S)-2-amino-3-(5-hydroxy-1H-indol-3-yl)propenoic acid: | 5-OHC₆H₃*C(CHC(NH₂)COOH)CHNH |
| d) [11-13C]-(S)-2-amino-3-(5-hydroxy-1H-indol-3-yl)propenoic acid: | 5-OHC₆H₃C(CHC(NH₂)*COOH)CHNH |
| e) [13C₆]-(S)-2-amino-3-(5-hydroxy-1H-indol-3-yl)propenoic acid: | 5-OH*C₆H₃C(CHC(NH₂)COOH)CHNH(phenyl-13C₆) |
| 36) (S)-2-amino-3-(3,4-dihydroxyphenyl)propenoic acid | |
| a) [7-13C]-(S)-2-amino-3-(3,4-dihydroxyphenyl)propenoic acid: | 3-HO—,4HO—C₆H₃*CHC(NH₂)COOH |
| b) [8-13C]-(S)-2-amino-3-(3,4-dihydroxyphenyl)propenoic acid: | 3-HO—,4HO—C₆H₃CH*C(NH₂)COOH |
| c) [9-13C]-(S)-2-amino-3-(3,4-dihydroxyphenyl)propenoic acid: | 3-HO—,4HO—C₆H₃CHC(NH₂)*COOH |
| d) [13C₆]-(S)-2-amino-3-(3,4-dihydroxyphenyl)propenoic acid: | 3-HO—,4HO—*C₆H₃CHC(NH₂)COOH(phenyl-13C₆) |
| e) [7,8-13C]-(S)-2-amino-3-(3,4-dihydroxyphenyl)propenoic acid: | 3-HO—,4HO—C₆H₃*CH*C(NH₂)COOH |
| f) [7,8,9-13C]-(S)-2-amino-3-(3,4-dihydroxyphenyl)propenoic acid: | 3-HO—,4HO—C₆H₃*CH*C(NH₂)*COOH |
| g) [13C₉]-(S)-2-amino-3-(3,4-dihydroxyphenyl)propenoic acid: | 3-HO—,4HO—*C₆H₃*CH*C(NH₂)*COOH |
| 37) L-Arginine | |
| a) [1-13C]-arginine: | ⁺NH₂C(NH₂)NHCH₂CH₂CH₂CH(NH₂)*CO₂H |
| b) [2-13C]-arginine: | ⁺NH₂C(NH₂)NHCH₂CH₂CH₂*CH(NH₂)CO₂H |
| c) [6-13C]-arginine: | ⁺NH₂*C(NH₂)NHCH₂CH₂CH₂CH(NH₂)CO₂H |
| 38) L-Citrulline | |
| a) [1-13C]-citrulline: | NH₂CONHCH₂CH₂CH₂CH(NH₂)*CO₂H |
| b) [2-13C]-citrulline: | NH₂CONHCH₂CH₂CH₂*CH(NH₂)CO₂H |
| c) [6-13C]-citrulline: | NH_{2*}CONHCH₂CH₂CH₂CH(NH₂)CO₂H |
| 39) 2-amino-2-ene-5-(diaminomethylidene amino)pentanoic acid | |
| a) [1-13C]-2-amino-2-ene-5-(diaminomethylidene amino)pentanoic acid: | ⁺NH₂C(NH₂)NHCH₂CH₂CHC(NH₂)*CO₂H |
| b) [2-13C]-2-amino-2-ene-5-(diaminomethylidene amino)pentanoic acid: | ⁺NH₂C(NH₂)NHCH₂CH₂CH*C(NH₂)CO₂H |
| c) [6-13C]-2-amino-2-ene-5-(diaminomethylidene amino)pentanoic acid: | ⁺NH₂*C(NH₂)NHCH₂CH₂CHC(NH₂)CO₂H |
| 40) 2-amino-5-(diaminomethylidene imino)pentanoic acid | |
| a) [1-13C]-2-amino-5-(diaminomethylidene imino)pentanoic acid | ⁺NH₂C(NH₂)NCHCH₂CH₂CH(NH₂)*CO₂H |
| b) [2-13C]-2-amino-5-(diaminomethylidene imino)pentanoic acid | ⁺NH₂C(NH₂)NCHCH₂CH₂*CH(NH₂)CO₂H |
| c) [6-13C]-2-amino-5-(diaminomethylidene imino)pentanoic acid | ⁺NH₂*C(NH₂)NCHCH₂CH₂CH(NH₂)CO₂H |

It is apparent to those of the skill that due to limitations imposed by synthesis procedures other labeled derivatives might have the same magnetic resonance activity. For example, labeled agents such as detailed above with additional carbon-13 label at another position or an additional nitrogen-15 nucleus at another position or with less labeled positions. These derivatives are included in the current invention.

### BRIEF DESCRIPTION OF THE DRAWINGS

In order to understand the invention and to see how it may be implemented in practice, a plurality of embodiments will now be described, by way of non-limiting example only, with reference to the accompanying drawings, in which:

FIG. 1. Molecular structure and assignment of labeled positions in choline;

FIG. 2. Molecular structure and assignment of labeled positions in betaine;

FIG. 3. Molecular structure and assignment of labeled positions in acetylcholine;

FIG. 4. Molecular structure and assignment of labeled positions in acetate;

FIG. 5. Molecular structure and assignment of labeled positions in aspartate;

FIG. 6. Molecular structure and assignment of labeled positions in N-acetylaspartate;

FIG. 7. Molecular structure and assignment of labeled positions in creatine;

FIG. 8. Molecular structure and assignment of labeled positions in L-tyrosine;

FIG. 9. Molecular structure and assignment of labeled positions in L-DOPA;

FIG. 10. Molecular structure and assignment of labeled positions in dopamine;

FIG. 11. Molecular structure and assignment of labeled positions in norepinephrine;

FIG. 12. Molecular structure and assignment of labeled positions in epinephrine;

FIG. 13. Molecular structure and assignment of labeled positions in vanillylmandelic acid;

FIG. 14. Molecular structure and assignment of labeled positions in homovanillic acid;

FIG. 15. Molecular structure and assignment of labeled positions in 3-O-methyldopamine;

FIG. 16. Molecular structure and assignment of labeled positions in 3-O-methylnorepinephrine;

FIG. 17. Molecular structure and assignment of labeled positions in 3-O-methylepinephrine;

FIG. 18. Molecular structure and assignment of labeled positions in dopaquinone;

FIG. 19. Molecular structure and assignment of labeled positions in L-tryptophan;

FIG. 20. Molecular structure and assignment of labeled positions in 5-hydroxy-tryptophan;

FIG. 21. Molecular structure and assignment of labeled positions in serotonin;

FIG. 22. Molecular structure and assignment of labeled positions in 5-hydroxyindole acetaldehyde;

FIG. 23. Molecular structure and assignment of labeled positions in 5-hydroxyindole acetic acid;

FIG. 24. Molecular structure and assignment of labeled positions in melatonin;

FIG. 25. Molecular structure and assignment of labeled positions in glutamate;

FIG. 26. Molecular structure and assignment of labeled positions in gamma-aminobutyric acid;

FIG. 27. Molecular structure and assignment of labeled positions in rivastigmine tartrate;

FIG. 28. Molecular structure and assignment of labeled positions in rasagiline;

FIG. 29. Molecular structure and assignment of labeled positions in methylphenidate;

FIG. 30. Molecular structure and assignment of labeled positions in amphetamine;

FIG. 31. Molecular structure and assignment of labeled positions in imidazopyridinylbenzeneamine derivatives;

FIG. 32. Molecular structure and assignment of labeled positions in benzothizolylbenzeneamine derivatives;

FIG. 33. Molecular structure and assignment of labeled positions in (2-hydroxyethenyl) trimethylammonium;

FIG. 34. Molecular structure and assignment of labeled positions in (2-hydroxyethynyl) trimethylammonium;

FIG. 35. Molecular structure and assignment of labeled positions in (S)-2-amino-3-(5-hydroxy-1H-indol-3-yl)propenoic acid;

FIG. 36. Molecular structure and assignment of labeled positions in (S)-2-amino-3-(3,4-dihydroxyphenyl)propenoic acid;

FIG. 37. Molecular structure and assignment of labeled positions in arginine;

FIG. 38. Molecular structure and assignment of labeled positions in citrulline;

FIG. 39. Molecular structure and assignment of labeled positions in 2-amino-2-ene-5-(diaminomethylidene amino)pentanoic acid;

FIG. 40. Molecular structure and assignment of labeled positions in 2-amino-5-(diaminomethylidene imino)pentanoic acid

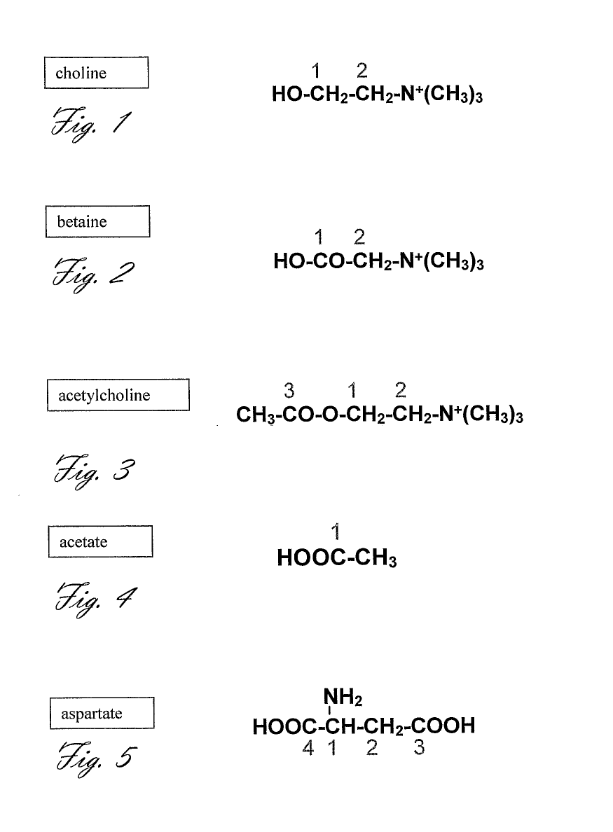

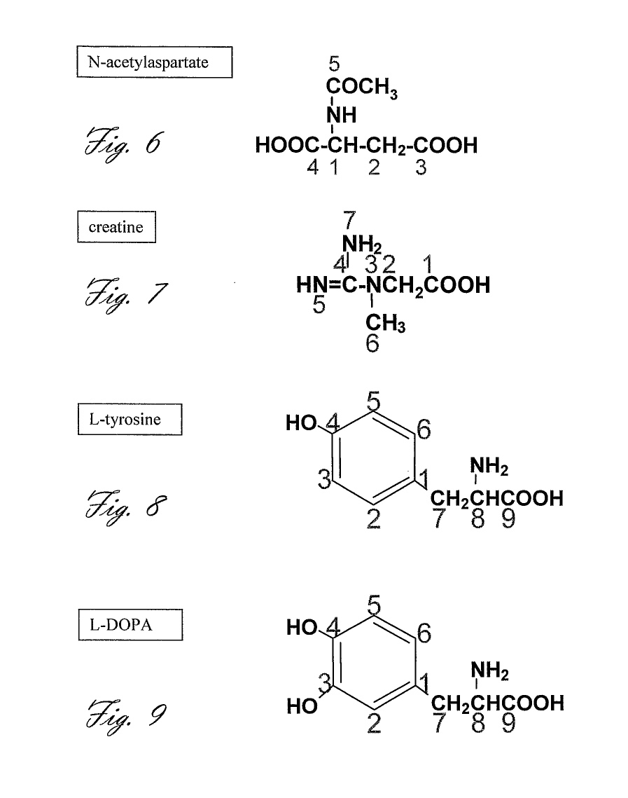

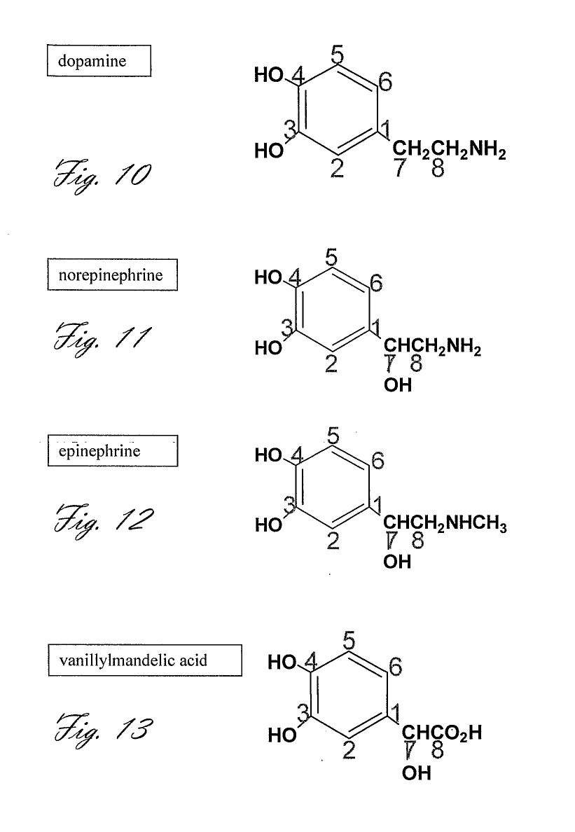

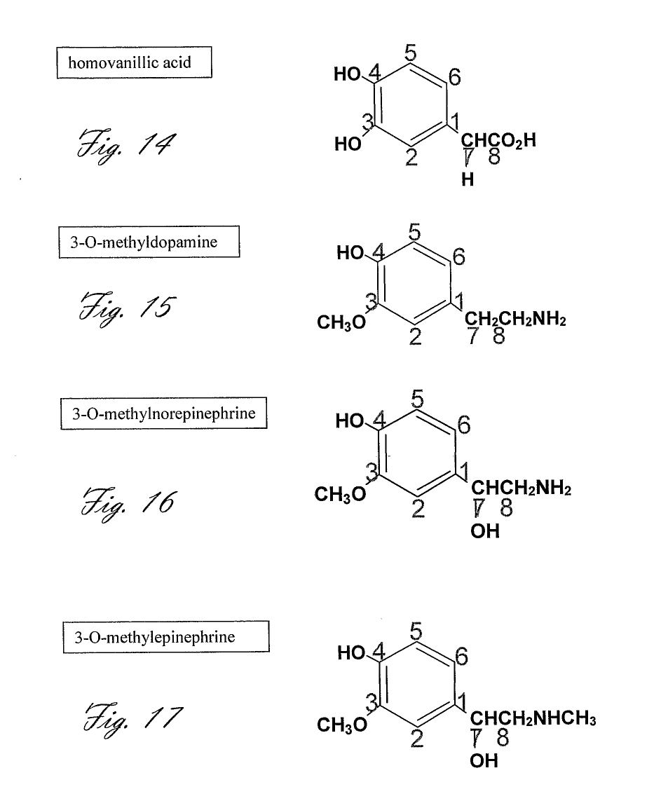

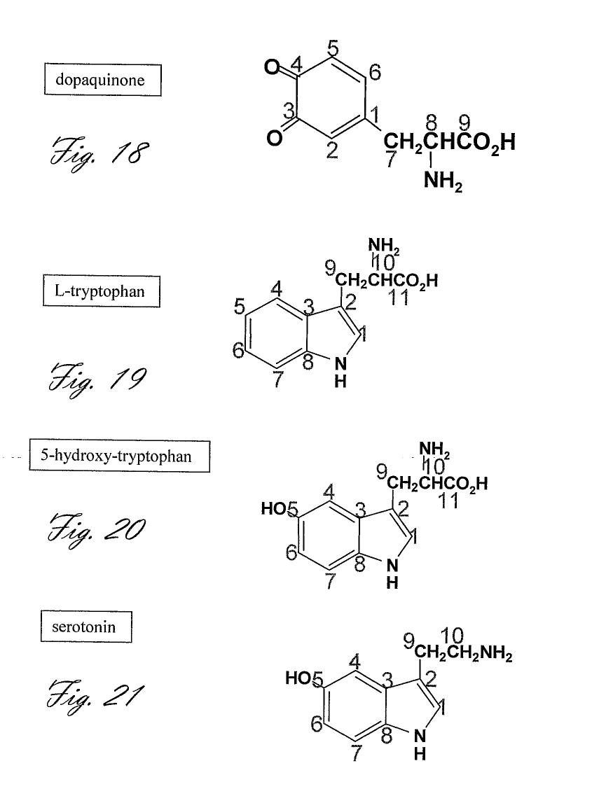

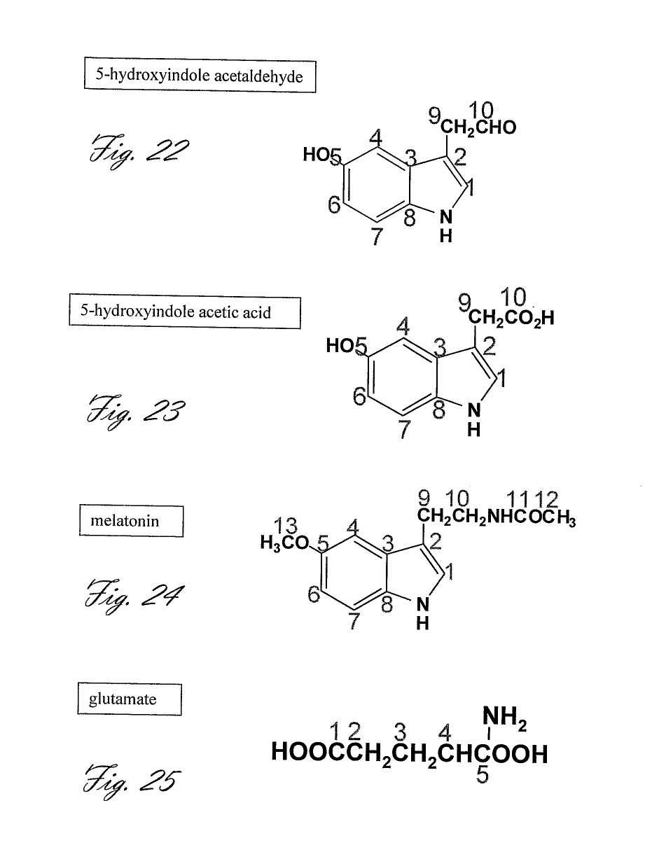

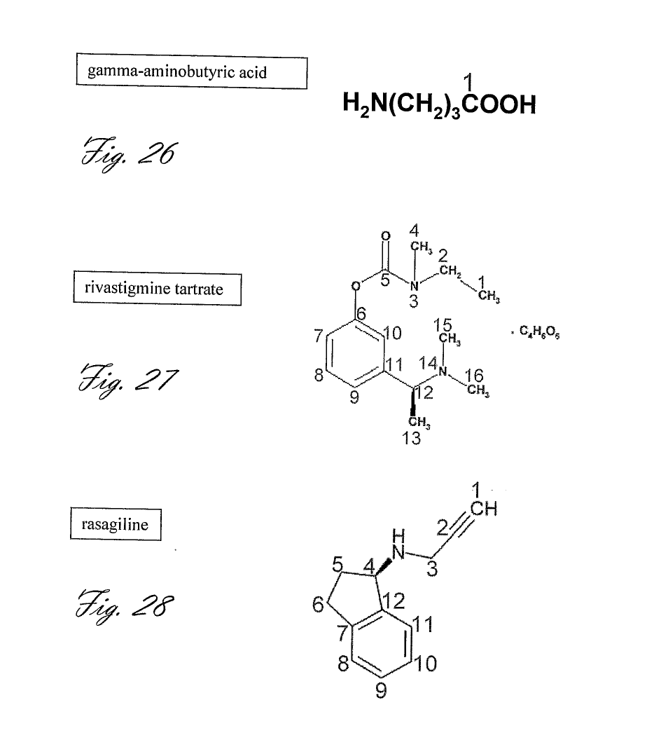

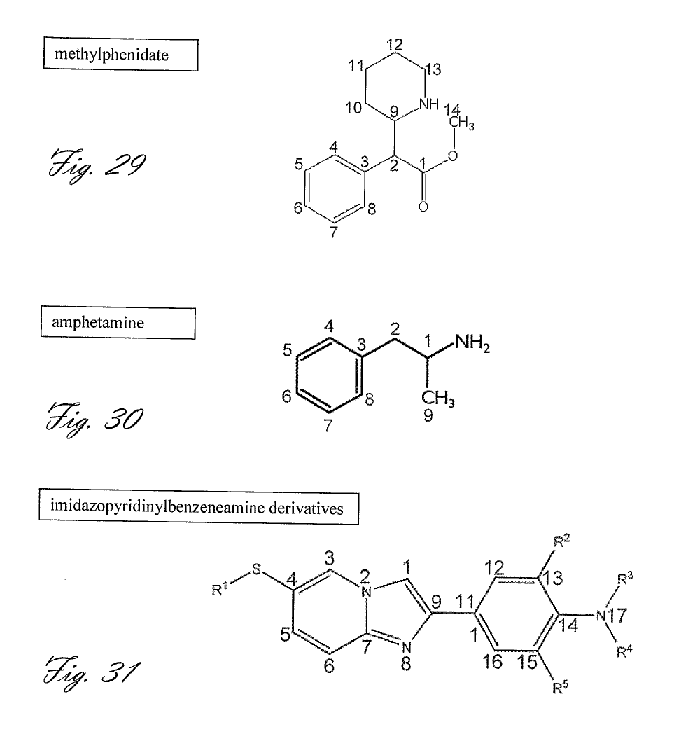

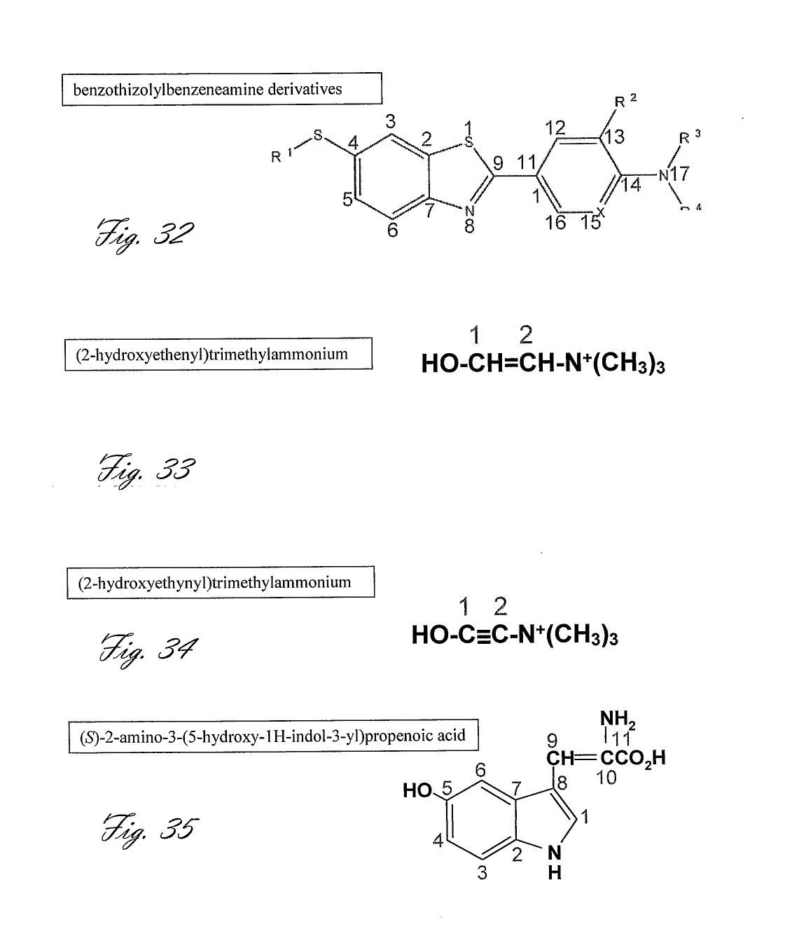

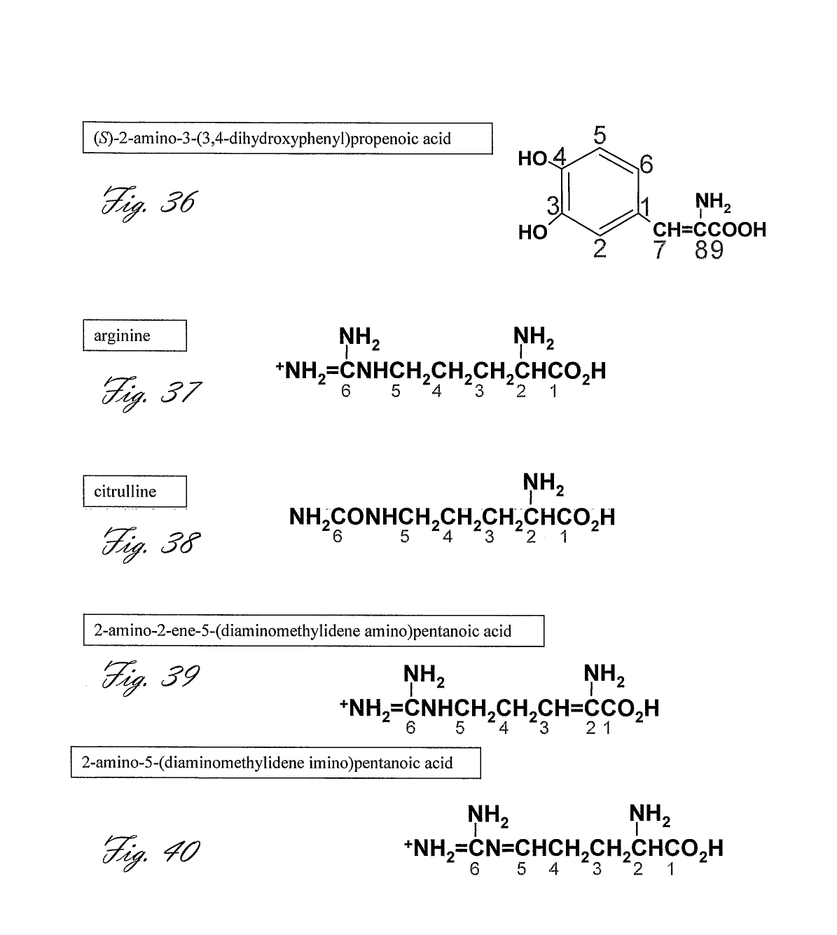

### DETAILED DESCRIPTION OF THE EMBODIMENTS

Ex vivo polarization may be carried out by any known method and by way of example two such methods are described herein below. It is envisaged that, in the method according to the invention, the level of polarization achieved should be sufficient to allow the HTNC agent to achieve a diagnostically effective contrast enhancement in the sample to which it is subsequently administered in whatever form. In general, it is desirable to achieve a level of polarization which is at least a factor of 2 or more above the field in which MRI is performed, preferably a factor of 10 or more, particularly preferably 100 or more and especially preferably 1000 or more, 10000 or more, and 100000 or more.

### Ex-Vivo Polarization—Method 1:

Ex vivo polarization of the MR imaging nuclei is effected by an OMRI contrast agent. This approach comprises two major steps: 1. bringing an OMRI contrast agent and a HTNC agent into contact in a uniform magnetic field (the primary magnetic field B₀); and 2. exposing said OMRI contrast agent to a first radiation of a frequency selected to excite electron spin transitions in said OMRI contrast agent.

For the purposes of administration, the high HTNC agent should be preferably administered in the absence of the whole of, or substantially the whole of, the OMRI contrast agent. Preferably at least 80% of the OMRI contrast agent is removed, particularly preferably 90% or more, especially preferably 95% or more, most especially 99% or more. In general, it is desirable to remove as much of the OMRI contrast agent as possible prior to administration to improve physiological tolerability and to increase T₁. Thus preferred OMRI contrast agents for use are those which can be conveniently and rapidly separated from the polarized HTNC agent. Such OMRI contrast agents are known in the art and may be employed for this purpose. However where the OMRI contrast agent is non-toxic, the separation step may be omitted. A solid (e.g. frozen) composition comprising an OMRI contrast agent and a HTNC agent which has been subjected to polarization may be rapidly dissolved in saline (e.g. warm saline) and the mixture injected shortly thereafter.

### Ex-Vivo Polarization—Method 2:

Generally speaking, polarization of an MR imaging nuclei within the HTNC may be achieved by thermodynamic equilibration at low temperature and high magnetic field.

Where the contrast medium to be administered is a solid material (e.g. crystalline), it may be introduced into a magnetic field at very low temperature. In this case, an OMRI contrast agent is not involved and there is no need for any separation process. Therefore, the polarized HTNC can be administered into the body or brain immediately after polarization.

### Ex-Vivo Polarization—Method 3:

Ex-vivo polarization is effected by hydrogenation of an unsaturated bond in the HTNC molecule by parahydrogen. This approach comprises 3 major steps: 1) production of parahydrogen, 2) hydrogenation of the unsaturated bond with parahydrogen in the presence of a hydrogenation catalyst, and 3) field cycling for transferring the increased spin order from protons to the carbon-13 nuclei.

For the purposes of administration, the high HTNC agent should be preferably administered in the absence of the whole of, or substantially the whole of, the hydrogenation catalyst. Preferably at least 80% of the hydrogenation catalyst is removed, particularly preferably 90% or more, especially preferably 95% or more, most especially 99% or more. In general, it is desirable to remove as much hydrogenation catalyst as possible prior to administration to improve physiological tolerability. Thus preferred hydrogenation catalysts for use are those which can be conveniently and rapidly-separated from the polarized HTNC agent. Such hydrogenation catalysts are known in the art and may be employed for this purpose. However where the hydrogenation catalyst is non-toxic, the separation step may be omitted.

The HTNC agents used in the method according to the invention may be conveniently formulated with conventional pharmaceutical or veterinary carriers or excipients. Formulations manufactured or used according to this invention may contain, besides the HTNC agent, formulation aids such as are conventional for therapeutic and diagnostic compositions in human or veterinary medicine. Thus the formulation may for example include stabilizers, antioxidants, osmolality adjusting agents, solubilizing agents, emulsifiers, viscosity enhancers, buffers, etc. The formulation may be in forms suitable for parenteral (e.g. intravenous or intraarterial) or enteral (e.g. oral) administration. However solutions, suspensions and dispersions in physiological tolerable carriers e.g. water or saline will generally be preferred.

The formulation, will preferably be substantially isotonic and may conveniently be administered at a concentration sufficient to yield a 1 micromolar to 100 mM concentration of the HTNC agent in the investigated zone; however the precise concentration and dosage will of course depend upon a range of factors such as toxicity, the regional targeting ability of the HTNC agent and the administration route. The optimum concentration for the MR imaging or spectroscopic agent represents a balance between various factors. Formulations for intravenous or intraarterial administration would preferably contain the HTNC agent in concentrations of 1 mM to 10M, especially more than 50 mM, preferably more than 200 mM, more preferably more than 500 mM. Parenterally administrable forms should of course be sterile and free from physiologically unacceptable agents, and should have low osmolality to minimize irritation or other adverse effects upon administration and thus the formulation should preferably be isotonic or slightly hypertonic. Suitable vehicles include aqueous vehicles customarily used for administering parenteral solutions such as Sodium Chloride solution, Ringer's solution, Dextrose solution, Dextrose and Sodium Chloride solution, Lactated Ringer's solution and other solutions such as are described in Remington's Pharmaceutical Sciences, 15th ed., Easton: Mack Publishing Co., pp. 1405-1412 and 1461-1487 (1975) and The National Formulary XIV, 14th ed. Washington: American Pharmaceutical Association (1975). The compositions can contain preservatives, antimicrobial agents, buffers and antioxidants conventionally used for parenteral solutions, excipients and other additives which are compatible with the HTNC agents and which will not interfere with the manufacture, storage or use of the products.

The dosages of the HTNC agent used according to the method of the present invention will vary according to the precise nature of the HTNC agents used, the tissue of interest, and the measuring apparatus. Preferably the dosage should be kept as low as possible while still achieving a detectable contrast effect. In general, the maximum dosage will depend on toxicity constraints.

The invention is illustrated by the following Examples in a non-limiting manner:

### Example 1

### Acetylcholine Synthesis in the Brain

The subject is pretreated with atropine prior to choline injection to prevent cholinergic intoxication.

[2-¹³C, ¹⁵N]-choline (99% ¹³C-labeled, 99% ¹⁵N-labeled 10 mg) is dissolved in 40 mg of 50:50 glycerol:H₂O. The trityl radical (Tris{8-carboxyl-2,2,6,6-tetra[2-(1-hydroxyethyl)]-benzo(1,2-d:4,5-d′)bis(1,3)dithiole-4-yl}methyl sodium salt) is added to reach concentrations of either 15 or 20 mM. The mixture is placed in an open top chamber.

The mixture is polarized by microwaves for at least one hour at a field of 2.5 T at a temperature of 4.2 K (or lower 1.2 K). The progress of the polarization process is followed by in situ NMR recording, according to previously published procedure (Ardenkjaer-Larsen, J. (2001) U.S. Pat. No. 6,278,893).

When a suitable level of polarization has been reached, the chamber is rapidly removed from the polarizer and, while handled in a magnetic field of no less than 50 mT, the contents are quickly discharged and dissolved in warm saline (40° C., 5 ml).

The solution containing the polarized [2-¹³C, ¹⁵N]-choline (5 ml, the HTNC) is injected to the subject via intravenous catheter that is placed in advance.

The hyperpolarized solution is followed by 20 ml of saline or another routine wash-volume.

### Experiment 1

Step 1) An anatomic image of the brain is recorded beforehand and the location of the hippocampus is prescribed.

Step 2) One s, or 2 s, or 3 s, or 4 s, or 5 s, or 6 s, or 10 s, or 15 s, or 20 s, or 40 s, or 60 s after injection, a carbon-13 spectrum is recorded from a 1×1×1 cm³ (or 0.5×0.5×0.5 cm³, or 0.2×0.2×0.2 cm³, or 2×2×2 cm³), voxel (single voxel spectroscopy) located at the subject's hippocampus.

The spectroscopic investigation uses the point resolved spectroscopy (PRESS) sequence with short echo time (5, or 15, or 30 msec). Proton decoupling is applied during data acquisition.

Alternatively, it is known in the art that polarization can be transferred from the nitrogen-15 nucleus (which is also hyper-polarized at the end of the polarization process) to the neighboring carbon-13 nuclei, prior to data acquisition.

Step 3) The spectrum is Fourier transformed and the level of [2-¹³C, ¹⁵N]-choline and [2-¹³C, ¹⁵N]-acetylcholine in the subject's hippocampus is quantified. Other potential metabolic products of [2-¹³C, ¹⁵N]-choline such as [2-¹³C, ¹⁵N]-betaine, and [2-¹³C, ¹⁵N]-phosphocholine are quantified as well, simultaneously.

### Experiment 2

Step 1) and step 2) are the same as in experiment 1.

Step 2) is repeated every 100 msec, or every 200 msec, or every 300 msec or every 500 msec, or every 600 msec, or every 700 msec, or every 800 msec, or every 900 msec, or every 1 sec, or every 1.5 sec, or every 2 sec, or every 3 sec or every 4 sec.

Step 3) The spectra are Fourier transformed and the level of [2-¹³C, ¹⁵N]-choline and [2-¹³C, ¹⁵N]-acetylcholine in the subject's hippocampus at each time point is quantified. Kinetic data of [2-¹³C, ¹⁵N]-choline accumulation and [2-¹³C, ¹⁵N]-acetylcholine synthesis are calculated, taking into account polarization decay, blood flow, and the kinetics of choline transport across the blood-brain-barrier.

### Experiment 3

Experiment 1 or 2 are repeated at a different location in the brain, for example the frontal lobe.

### Experiment 4

Experiments 1 or 2 or 3 are performed, with step 2 including a spectroscopic imaging sequence, sampling a slice in the brain at a selected level. The in plane resolution of the spectroscopic image is 0.2 cm, or 0.4 cm, or 0.5 cm, 1 cm, 2 cm, or 3 cm.

The slice thickness is 0.2 cm, or 0.4 cm, or 0.5 cm, or 1 cm, 2 cm, 5 cm, or 10 cm.

Alternatively, a multislice spectroscopic imaging sequence can be applied to sample the entire brain.

### Experiment 5

Experiments 1 or 2 or 3 or 4 are performed on a group of 10, or 50, or 100 animals (for example, rats, rabbits, mini-pigs, pigs).

The experiment is repeated on the same group of animals (a few days later) or on a different group of animals, this time while the animals receive a drug that is aimed at modifying the acetylcholine level in the brain, for example, a novel or well-known acetylcholine esterase inhibitor therapy.

The individual and the average rate of choline uptake and acetylcholine synthesis in the normal animal brain are calculated, and drug efficacy is determined.

### Experiment 6

Experiments 1 or 2 or 3 or 4 are performed on a group of 10, or 50, or 100, or 200, or 500 healthy volunteers who have no indication of a neurologic or psychiatric disorders and no history or current drug addiction or use.

The individual and the average rate of choline uptake and acetylcholine synthesis in the normal human brain are calculated. The maximal level of synthesized acetylcholine is determined as well.

The same experiment is performed in a group of patients who are diagnosed with mild cognitive impairment or various degrees of Alzheimer's disease who are not medicated.

The individual and the average rate of choline uptake and acetylcholine synthesis in the brain within this group of patients are calculated. The maximal level of synthesized acetylcholine in these patients is determined as well.

The same experiment is performed in a group of patients who are receiving a novel drug treatment or an existing acetylcholine esterase inhibitor drug treatment (such as rivastigmine).

The individual and the average rate of choline uptake and acetylcholine synthesis in the brain within this group of treated patients are calculated.

By comparison, the drug efficacy in individuals as well as in groups of patients can be determined. Individuals can be monitored routinely at reasonable time durations to confirm continued treatment effectiveness.

### Experiment 7

Experiments 1 or 2 or 3 or 4 are performed in the same subject or patient, several times trough the day and night, to determine patterns of choline transport and acetylcholine synthesis. The individual's pattern of acetylcholine synthesis and release is used to design an individualized schedule of controlled acetylcholine release from a controlled release device that is implanted in the subject's brain or a controlled release of choline into the brain or circulation.

### Experiment 8

Experiments 1, or 2, or 3, or 4 are performed in a patient that has been diagnosed with a brain tumor. The level and rate of [2-¹³C, ¹⁵N]-choline transport, [2-¹³C, ¹⁵N]-phosphocholine synthesis, and [2-¹³C, ¹⁵N]-betaine synthesis in the investigated tissue aid in the characterization of the tumor or the malignant potential at the tissue surrounding the tumor, as it is known in the art that choline metabolism is altered in malignant tissues. An extension of this experiment is the characterization of tumors in the body, such as tumors in the breast, prostate, and kidney.

### Example 2

### Dopamine Synthesis in the Brain

[¹³C₆]-L-DOPA (99% ¹³C-labeled phenyl, 10 mg) is hyperpolarized and dissolved according to the procedure described in Example 1.

The subject is pretreated with a single dose or several doses of aromatic-L-amino-acid decarboxylase inhibitor such as carbidopa or benserazide, or difluoromethyldopa, or α-methyldopa (20 mg, 40 mg, 60 mg, or 80 mg) given orally.

1 hour after pretreatment with carbidopa, the hyperpolarized solution (cooled to 37° C.), is quickly injected to the subject (preferably in less than 10 sec, or as described in Example 1).

### Experiment 1

Step 1) Similar to Example 1, Experiment 1, Step 1.

Step 2) Similarly to Example 1, Experiment 1, Step 2, carbon-13 magnetic resonance spectra are recorded from a single volume element located at a specific location such as the substantianigra, striatum, basal ganglia, or the thalamus of the subject.

Step 3) The spectra are Fourier transformed and the levels of [¹³C₆]-L-DOPA, [¹³C₆]-dopamine, [¹³C₆]-homovanillic acid, and [¹³C₆]-3-O-methyldopamine and other potential metabolic products of [¹³C₆]-L-DOPA, at the specific location, are quantified, simultaneously.

### Experiment 2

Repeated measurements of the types that are described in Experiment 1, and kinetic analysis as described in Example 1, Experiment 2.

### Experiment 3

Spectroscopic imaging of the distribution of [¹³C₆]-L-DOPA, [¹³C₆]-dopamine, and other potential metabolites of [¹³C₆]-L-DOPA, as described in Example 1, Experiment 4.

### Experiment 4

Experiments 1 or 2 or 3 are performed on a group of 10, or 50, or 100 animals (for example, rats, rabbits, mini-pigs, pigs).

The experiment is repeated on the same group of animals (a few days later) or on a different group of animals, this time while the animals receive a drug that is aimed at increasing the dopamine level in the brain, for example, a novel or a well-known monoamine oxidase inhibitor therapy.

The level of [¹³C₆]-dopamine and other [¹³C₆]-L-DOPA metabolites in the brain is determined in both groups of animals. The individual and the average rate of [¹³C₆]-L-DOPA uptake and [¹³C₆]-dopamine synthesis in the naive and treated brain are calculated, and drug efficacy is determined.

### Experiment 5

Experiments 1 or 2 or 3 are performed on a group of 10, or 50, or 100, or 200, or 500 healthy volunteers who have no indication of a neurologic or psychiatric disorders and no history or current drug addiction or use.

The level of [¹³C₆]-dopamine and other [¹³C₆]-L-DOPA metabolites in the normal human brain is determined. The individual and the average rate of [¹³C₆]-L-DOPA uptake and [¹³C₆]-dopamine synthesis in the normal human brain are calculated.

The same experiment is performed in a group of patients who are diagnosed with Parkinson's disease and who are not medicated.

The level of [¹³C₆]-dopamine and other [¹³C₆]-L-DOPA metabolites in the brain of patients with Parkinson's disease is determined. The individual and the average rate of [¹³C₆]-L-DOPA uptake and [¹³C₆]-dopamine synthesis in the brain within this group of patients are calculated.

The same experiment is performed in a group of patients who are receiving a novel or well-known monoamine oxidase inhibitor drug treatment (such as rasagiline).

The level of [¹³C₆]-dopamine and other [¹³C₆]-L-DOPA metabolites in the treated patients is determined. The individual and the average rate of [¹³C₆]-L-DOPA uptake and [¹³C₆]-dopamine synthesis in the treated patients are calculated.

By comparison, the drug efficacy in individuals as well as in groups of patients can be determined. Individuals can be monitored routinely within reasonable time duration to insure drug effectiveness.

### Experiment 6

Experiments 1 or 2 or 3 are performed in the same subject or patient, several times trough the day and night, to determine patterns of L-DOPA uptake and dopamine synthesis in the individual's brain. The data are used to design a schedule of controlled release of L-DOPA, dopamine, or a drug such as monoamine oxidase inhibitor, from a controlled release device that is implanted in the subject's brain or a controlled release of L-DOPA and carbidopa into the circulation.

Alternatively, if deep brain stimulation (DBS) is being considered as a therapeutic route, the data are used to aid in determination of the best location for placing DBS electrodes.

### Experiment 7

[¹³C₆]-(S)-2-amino-3-(3,4-dihydroxyphenyl)propenoic acid_(99% ¹³C-labeled phenyl, 10 mg) is hydrogenated with parahydrogen in the presence of a hydrogenation catalyst or an asymmetric hydrogenation catalyst. The hydrogenation catalyst is separated from the DOPA product using a filtration column, or molecular size sieve, or phase separation (DOPA is more hydrophilic that most catalysts), within a few seconds. Where both D- and L enantiomers of DOPA are produced, they may be quickly separated (in less than 5 sec). The [¹³C₆]-L-DOPA solution is undergoing magnetic field cycling to transfer the polarization to the ¹³C nuclei.

The subject is pretreated with a single dose or several doses of aromatic-L-amino-acid decarboxylase inhibitor such as carbidopa or benserazide, or difluoromethyldopa, or α-methyldopa (20 mg, 40 mg, 60 mg, or 80 mg) given orally.

1 hour after pretreatment with carbidopa, the hyperpolarized [¹³C₆]-L-DOPA_solution (5 ml, the HTNC) is quickly injected to the subject (preferably in less than 10 sec, or as described in Example 1), via intravenous catheter that is placed in advance. The hyperpolarized solution is followed by 20 ml of saline or another routine wash volume. Experiments 1 through 6 in this example (example 2) are performed. The HTNC is the same in both cases; the difference in experiment 7 is that the hyperpolarization step was achieved via PHIP instead of DNP.

### Example 3

### Dopamine

### Acetylcholine Balance in the Brain

The subject is pretreated with atropine and carbidopa as described in Examples 1 and 2. [¹³C₆]-L-DOPA (99% ¹³C-labeled phenyl, 10 mg) and [2-¹³C, ¹⁵N]-choline (99% ¹³C-labeled, 99% ¹⁵N-labeled 10 mg) are hyperpolarized and dissolved according to the procedure described in Example 1.

The hyperpolarized solution (cooled to 37° C.), is quickly injected to the subject (preferably in less than 10 sec, or as described in Example 1).

The solution containing the polarized [¹³C₆]-L-DOPA and [2-¹³C, ¹⁵N]-choline (5 ml, the HTNC) is injected to the subject via intravenous catheter that is placed in advance.

The hyperpolarized solution is followed by 20 ml of saline or another routine wash volume.

The balance between acetylcholine production and dopamine production and metabolism is quantified in animal models and in the human brain using the experiments that are described above. Specifically, the effects of existing and novel drugs on this balance is investigated and aids in determination of the drug course of action in situ and drug efficacy.

### Example 4

### Serotonin Level and Metabolism in the Brain

[8-¹³C, ¹⁵N]-5-hydroxy-tryptophan (99% ¹³C-labeled, 10 mg, the HTNC) is hyperpolarized and dissolved according to the procedure described in Example 1.

Alternatively, the hyperpolarized HTNC is produced by PHIP of [8-13C]-(S)-2-amino-3-(5-hydroxy-1H-indol-3-yl)propenoic acid via hydrogenation with parahydrogen, in a similar manner to that described in Example 2, experiment 7.

At the end of the polarization process the hyperpolarized solution (cooled to 37° C.), is quickly injected to subject (preferably in less than 10 sec, or as described in Example 1). The uptake of [8-¹³C, ¹⁵N]-5-hydroxy-tryptophan and synthesis of [8-¹³C, ¹⁵N]-serotonin is monitored by carbon-13 magnetic resonance spectroscopy methods and experiments, as described above.

Alternatively, the level of these molecules and their potential metabolites is also monitored by nitrogen-15 magnetic resonance spectroscopy.

Alternatively, the total level of 5-hydroxy-tryptophan and its various metabolites is monitored by carbon-13 and nitrogen-15 imaging (without the chemical shift dimension). In this type of imaging, areas of strong signal indicates the presence of relatively high levels of 5-hydroxy-tryptophan and serotonin metabolites, and depending on the MRI sequence parameters, one could also differentiate between molecules that are located in the extracellular, intracellular, and intravesicular spaces.

The kinetics of 5-hydroxy-tryptophan uptake, serotonin synthesis, and further serotonin metabolism is characterized in situ in the brain using the methods and experimental procedures descried in examples 1 through 4.

These data are used to determine the effect of novel and existing serotonergic drugs such as selective serotonin reuptake inhibitors.

### Example 5

### Distribution of Specific Enzymatic Subtypes in the Brain

[2-13C]-rasagiline (99% enriched, 5 mg) is hyperpolarized and dissolved according to the procedure described in Example 1 or Example 2, experiment 7. The kinetics of uptake and possible metabolism of rasagiline in the brain are monitored by carbon-13 magnetic resonance spectroscopy using experimental procedures as described above.

Alternatively, the distribution of [2-13C]-rasagiline in the brain is monitored by magnetic resonance imaging (without the chemical shift dimension). Areas of high intensity in this image will indicate a high level of rasagiline in the area and, depending on the MRI sequence parameters, the physical state of rasagiline: bound, free, degree of freedom of motion, and surrounding medium chemistry and viscosity.

Interpretation of the results of this type of images is used to provide information on the levels of monoamine oxidase inhibitors in various areas in the brain. This information can be used for diagnosis and treatment monitoring of Parkinson's disease and Alzheimer's disease. This information is also important for strategic planning of the use of the drug in humans.

1-40. (canceled)

41. High T₁ neurochemical and biochemical contrast agents (HTNCs) for imaging metabolic processes and activities in the brain or body of either human or otherwise mammalian (patient); said HTNCs comprising chemicals involved in neuronal or brain function or neuromodulatory processes in the brain of said patient, vascular function, or organ specific metabolic processes; said HTNCs are labeled with stable isotopes selected from a group consisting of deuterium, carbon-13, nitrogen-15, fluorine-19 (²H, ¹³C, ¹⁵N, ¹⁹F) or a combination thereof in predetermined positions, so as to enhance the detectability of both the agents and their metabolic successors.

42. The HTNCs of claim 41, selected from a group consisting of:
a. molecules of metabolic processes selected from a group consisting of choline, betaine, acetylcholine, acetate, aspartate, N-acetylaspartate, creatine, L-tyrosine, L-DOPA, dopamine, norepinephrine, epinephrine, vanillylmandelic acid (VMA), homovanillic acid (HVA), 3-O-methyldopamine, 3-O-methylnorepinephrine, 3-O-methylepinephrine, dopaquinone, L-tryptophan, 5-hydroxy-tryptophan, serotonin, 5-hydroxyindole acetaldehyde, 5-hydroxyindole acetic acid, melatonin, glutamate, arginine, citrulline, N-acetylcitrulline, argininosuccinate, kynurenic acid (KYNA), 7-chlorokynurenic acid (7-Cl—KYNA), kynurenine, 4-chlorokynurenine, pharmacologically acceptable salts thereof, or any combination thereof;
b. molecules used in drugs selected from a group consisting of psychiatric or neuroprotective drugs, blood flow modulating drugs, mood altering drugs; with and drugs selected from a group consisting of rivastigmine, rasagiline, methylphenidate, amphetamine, tacrine, donepezil, metrifonate, fluoxetine, sertraline, paroxetine, fluvoxamine, citalopram, escitalopram, venlafaxine, nefazodone, mirtazapine, bupropion, cianopramine, femoxetine, ifoxetine, milnacipran, oxaprotiline, sibutramine, viqualine, clozapine, fenclonine, dexfenfluramine, chlorpromazine, methamphetamine, prazosin, terazosin, doxazosin, trimazosin, labetalol, medroxalol, tofenacin, trazodone, viloxazine, riluzole, pharmacologically acceptable salts thereof, or any combinations thereof being preferable;
c. molecules used as either PET or SPECT contrast agents; with molecules selected from a group consisting of ligands for dopamine receptors and transporters, serotonin receptors and transporters, acetylcholine receptors and transporters, norepinephrine receptors and transporters, beta-amyloid peptide and its imidazopyridinylbenzeneamine and benzothizolylbenzeneamine derivatives ligands, pharmacologically acceptable salts thereof, or any combinations thereof being preferable; and,
d. molecules that upon hydrogenation yield said HTNCs; with molecules selected from a group consisting of (2-hydroxyethenyl)trimethylammonium, (2-hydroxyethynyl) trimethylammonium, (S)-2-amino-3-(5-hydroxy-1H-indol-3-yl)propenoic acid, (S)-2-amino-3-(3,4-dihydroxyphenyl)propenoic acid, 2-amino-2-ene-5-(diaminomethylidene amino) pentanoic acid, 2-amino-5-(diaminomethylidene imino)pentanoic acid, pharmacologically acceptable salts thereof, or any combination thereof being preferable.

43. The HTNCs of claim 41 and 42, comprising at least one nucleus with a T₁ value of at least 2 to 300 seconds, at a field strength of 0.01 to 5 Tesla and a temperature in the range of 20 to 40° C.

44. The HTNCs of claim 41 and 42, comprising at least one ¹³C nucleus in at least one particular position in its molecular structure in an amount above 1% and up to 100%, with 99% being preferable.

45. The HTNCs of claim 44, comprising at least one deuterium nucleus, being either adjacent or remote to said ¹³C nucleus, wherein said deuterated position is either (i) enriched in an amount higher or equal to 1%; or (ii) labeled with ¹⁹F.

46. The HTNCs of claim 41 and 42, comprising at least one ¹⁵N nucleus in at least one particular position in its molecular structure, wherein said ¹⁵N position is enriched in an amount above 1% and up to 100%, with 99% enrichment being preferable.

47. A method of detecting spatial and temporal distribution of High T₁ neurochemical and biochemical contrast agents (HTNCs) and their metabolic/catabolic products within the brain or body of either human or mammalian (patient); said method comprising at least one of the following steps:
a. ex vivo polarizing one or more HTNCs involved in neuronal or brain function, or neuromodulatory processes in the brain of said patient, vascular function, or organ specific metabolic processes; said HTNCs are labeled with stable isotopes selected from a group consisting of deuterium, carbon-13, nitrogen-15, fluorine-19 (²H, ¹³C, ¹⁵N, ¹⁹F) or a combination thereof in predetermined positions;
b. administrating a human, or otherwise a mammalian patient said polarized HTNCs; and,
c. monitoring the distribution of said HTNCs and their metabolic successors in the brain or body of said patients by means of magnetic resonance spectroscopy and imaging the same; said monitoring applied after at least one step of administrating of said polarized HTNCs, in at least one time point after said administration.

48. The method according to claim 47, further comprising steps selected from a group consisting of:
a. subjecting said HTNCs agents to ex vivo polarization, and where this is carried out by means of a polarizing agent or catalyst and polarization apparatus, optionally separating the whole, or a portion of said polarizing agent or catalyst from said HTNCs agents;
b. administering said HTNCs agents to the human or non-human mammalian patient body or brain;
c. exposing said body or brain to a radiation of a frequency selected to excite nuclear spin transitions in selected nuclei;
d. detecting magnetic resonance signals from said HTNCs and their metabolic/catabolic products within said body or brain of said patient;
e. optionally, generating images, metabolic data, enzyme kinetics data, transport kinetic data, diffusion data, relaxation data, or physiological data from said detected signals;
f. optionally, using the data obtained in step (e) to aid in quantifying neuronal function;
g. optionally, using the data obtained in step (f) to diagnose diseases and disorders of the brain;
h. optionally, using of the data obtained in steps (f) and (g) to monitor action of and response to therapy aimed at alleviating or curing psychiatric, neurodegenerative, and neurological diseases and disorders;
i. optionally, using the data obtained in step (f) to affirm drug activity in situ and determine drug efficacy;
j. optionally, using data obtained in step (f) for strategic planning of the location of deep brain stimulation electrodes and other neurostimulators;
k. optionally, using data obtained in step (f) for strategic planning for the location of slow-release or controlled release devices within the brain;
l. optionally, using data obtained in step (e) for characterization of masses, tumors, cysts, blood vessel abnormalities, and internal organ function; and,
m. optionally, using the data obtained in step (f) for evaluation and determination of the level of anesthesia, comatose states, and the brain regions affected by stroke or trauma and their penumbra;
wherein said HTNCs, in a solid form or in solution, comprising nuclei selected from the group consisting of ²H, ¹³C, ¹⁵N, and ¹⁹F nuclei; and further wherein said HTNCs are dissolved in an administrable media prior to administration to said human or mammalian patient

49. A method as claimed in claim 48, further comprising a step of providing said polarizing agent or catalyst, said polarizing agent or catalyst is in any state, including liquid state, solid state, or a combination thereof.

50. A method as claimed in claim 48, further comprising a step of providing an increase in the polarization of said HTNCs by at least two fold to 500,000 fold, compared to the thermal equilibrium polarization level of said HTNCs, such that the detectability of said HTNCs and their metabolic successors is enhanced.

51. The method of detecting spatial and temporal distribution of HTNCs and their metabolic/catabolic products within the brain or body of either human or mammalian (patient) of claims 47 or 48, further comprising a step of selecting said HTNCs from at least one group consisting of:
a. molecules of metabolic processes selected from a group consisting of choline, betaine, acetylcholine, acetate, aspartate, N-acetylaspartate, creatine, L-tyrosine, L-DOPA, dopamine, norepinephrine, epinephrine, vanillylmandelic acid (VMA), homovanillic acid (HVA), 3-O-methyldopamine, 3-O-methylnorepinephrine, 3-O-methylepinephrine, dopaquinone, L-tryptophan, 5-hydroxy-tryptophan, serotonin, 5-hydroxyindole acetaldehyde, 5-hydroxyindole acetic acid, melatonin, glutamate, arginine, citrulline, N-acetylcitrulline, argininosuccinate, kynurenic acid (KYNA), 7-chlorokynurenic acid (7-Cl—KYNA), kynurenine, and 4-chlorokynurenine, pharmacologically acceptable salts thereof, or any combination thereof;
b. molecules used in drugs selected from a group consisting of psychiatric or neuroprotective drugs, blood flow modulating drugs, mood altering drugs; with drugs selected from a group consisting of rivastigmine, rasagiline, methylphenidate, amphetamine, tacrine, donepezil, metrifonate, fluoxetine, sertraline, paroxetine, fluvoxamine, citalopram, escitalopram, venlafaxine, nefazodone, mirtazapine, bupropion, cianopramine, femoxetine, ifoxetine, milnacipran, oxaprotiline, sibutramine, viqualine, clozapine, fenclonine, dexfenfluramine, chlorpromazine, methamphetamine, prazosin, terazosin, doxazosin, trimazosin, labetalol, medroxalol, tofenacin, trazodone, viloxazine, riluzole, and pharmacologically acceptable salts thereof, or any combinations thereof being preferable;
c. molecules used as either PET or SPECT contrast agents; with molecules selected from a group consisting of ligands for dopamine receptors and transporters, serotonin receptors and transporters, acetylcholine receptors and transporters, norepinephrine receptors and transporters, beta-amyloid peptide and its imidazopyridinylbenzeneamine and benzothizolylbenzeneamine derivatives ligands, pharmacologically acceptable salts thereof, or any combinations thereof being preferable; and,
d. molecules that upon hydrogenation yield said HTNCs; with molecules selected from a group consisting of (2-hydroxyethenyl) trimethylammonium, (2-hydroxyethynyl) trimethylammonium, (S)-2-amino-3-(5-hydroxy-1H-indol-3-yl) propenoic acid, (S)-2-amino-3-(3,4-dihydroxyphenyl)propenoic acid, 2-amino-2-ene-5-(diaminomethylidene amino) pentanoic acid, 2-amino-5-(diaminomethylidene imino)pentanoic acid, pharmacologically acceptable salts thereof, or any combination thereof being preferable.

52. A system comprising of magnetic resonance scanner, polarizer, and software, wherein said system is adapted for detecting, analyzing, and quantifying the signals of the hyper-polarized HTNCs as defined in any of claims 41 or 42; said system is adapted to provide presentation of the metabolic results fused with the anatomic and functional images of the brain and body of a human or mammalian patient using image and spectra analysis.
